(19) United States
(12) Patent Application Publication
(10) Pub. No.: US 2004/0115184 A1
(43) Pub. Date: Jun. 17, 2004

(54) Methods and compositions for modifying apolipoprotein b mrna editing

(76) Inventors: Harold C Smith, South Rochester, NY (US); Yan Yang, Bar Harbor, ME (US); Mark P Sowden, Penfield, NY (US)

Correspondence Address: NEEDLE & ROSENBERG, P.C., SUITE 1000, 999 PEACHTREE STREET, ATLANTA, GA, 30309-3915, US

(21) Appl. No.: 10/468,987

(22) PCT Filed: Feb. 26, 2002
(86) PCT No.: PCT/US02/05824

(51) Int. Cl. A61K 38/48; C12N 9/12

(52) U.S. Cl. 424/94.5; 435/194

(57) ABSTRACT

Products and methods for modifying apolipoprotein B mRNA editing in vivo, reducing serum LDL levels, and treating or preventing an atherogenic disease or disorder are disclosed. Such methods involve the use of a protein including APOBEC-1 or fragments thereof which can edit mRNA encoding apolipoprotein B. The protein including APOBEC-1 can be taken up by cells in the form of a delivery vehicle, such as a liposome or noisome, or directly as a chimeric protein which includes a first polypeptide that includes a protein transduction domain and a second polypeptide that includes APOBEC-1 or a fragment thereof which can edit mRNA encoding apolipoprotein B.

This application claims the benefit of U.S. Provisional Patent Application Serial No. 60/271,856, filed Feb. 27, 2001, which is hereby incorporated by reference in its entirety.

This invention was made, at least in part, using funding received from the U.S. Public Health Service, grant DK43739. The U.S. government may have certain rights in this invention.

### FIELD OF THE INVENTION

The present invention related generally to the chimeric proteins, compositions and products containing one or more chimeric proteins, as well as the use thereof to modify apolipoprotein B processing, to treat or prevent atherogenic diseases or disorders, and to modify the intravascular lipoprotein population.

### BACKGROUND OF THE INVENTION

Cholesterol is carried in blood by specific carrier proteins called apolipoproteins and from one tissue to another as lipoprotein particles. Apolipoprotein B is an integral and non-exchangeable structural component of lipoprotein particles referred to as chylomicrons, very low density lipoprotein (“VLDL”), and low density lipoprotein (“LDL”). Apolipoprotein B circulates in human plasma as two isoforms, apolipoprotein B100 and apolipoprotein B48. Apolipoprotein B48 is generated by an RNA editing mechanism which changes codon 2153 (CAA) to a translation stop codon (UAA) (Chen et al., “Apolipoprotein B-48 is the product of a messenger RNA with an organ-specific in-frame stop codon,” Science 238:363-366 (1987); Powell et al., “A novel form of tissue-specific RNA processing produces apolipoprotein-B48 in intestine,” Cell 50:831-840 (1987)). Editing is a site-specific deamination event catalyzed by apolipoprotein B mRNA editing catalytic subunit 1 (known as APOBEC-1) (Teng et al., “Molecular cloning of an apo B messenger RNA editing protein,” Science 260:18116-1819 (1993)) with the help of auxiliary factors (Teng et al., “Molecular cloning of an apo B messenger RNA editing protein,” Science 260:18116-1819 (1993); Yang et al., “Partial characterization of the auxiliary factors involved in apo B mRNA editing through APOBEC-1 affinity chromatography,” J. Biol. Chem. 272:27700-27706 (1997); Yang et al., “Multiple protein domains determine the cell type-specific nuclear distribution of the catalytic subunit required for apo B mRNA editing,” Proc. Natl. Acad. Sci. USA 94:13075-13080 (1997); Lellek et al., “Purification and Molecular cloning of a novel essential component of the apo B mRNA editing enzyme complex,” J. Biol. Chem. 275:19848-19856 (2000); Mehta et al., “Molecular cloning of apobec-1 complementation factor, a novel RNA-binding protein involved in the editing of apolipoprotein B mRNA,” Mol. Cell. Biol. 20:1846-1854 (2000); Yang et al., “Induction of cytidine to uridine editing on cytoplasmic apolipoprotein B mRNA by overexpressing APOBEC-1,” J. Biol. Chem. 275:22663-22669 (2000); Blanc et al., “Identification of GRY-RBP as an apoB mRNA binding protein that interacts with both apobec-1 and with apobec-1 complementation factor (ACF) to modulate C to U editing,” J. Biol. Chem. 276:10272-10283 (2001)) as a holoenzyme or editosome (Smith et al. “In vitro apolipoprotein B mRNA editing: Identification of a 27S editing complex,” Proc. Natl. Acad. Sci. USA 88:1489-1493 (1991); Harris et al., “Extract-specific heterogeneity in high-order complexes containing apo B mRNA editing activity and RNA-binding proteins,” J. Biol. Chem. 268:7382-7392 (1993)). Apolipoprotein B100 and apolipoprotein B48 play different roles in lipid metabolism, most importantly, apolipoprotein B100-associated lipoproteins (VLDL and LDL) are much more atherogenic than apolipoprotein B48-associated lipoproteins (chylomicrons and their remnants and VLDL).

Specifically, the apolipoprotein B48-associated lipoproteins are cleared from serum more rapidly than the apolipoprotein B100-associated lipoproteins. As a result, apolipoprotein B48-VLDL usually are not present in serum for an amount of time sufficient for serum lipases to convert the VLDL to LDL. In contrast, the apolipoprotein B100-VLDL are present in the serum for sufficient amounts of time, allowing serum lipases to convert the VLDL to LDL. Elevated serum levels of LDL are of particular biomedical significance as they are associated with an increased risk of atherogenic diseases or disorders. Lipoprotein analyses have shown that the ability of mammalian liver to edit results in a lowering of the VLDL+LDL:HDL ratio. Therefore, it would be desirable to identify an approach for modifying apolipoprotein B editing which would favor an increase in the relative concentration of apolipoprotein B48 in proportion to apolipoprotein B100 (or total apolipoprotein concentration), thereby clearing a greater concentration of lipoproteins from serum and minimizing the atherogenic risks associated with high serum levels of VLDL and LDL.

Current lipid-lowering therapies include statins and bile-acid-binding resins. Statins are competitive inhibitors of hydroxymethylglutaryl-coenzyme A (HMG-CoA) reductase, which catalyzes the committed step in the synthesis of cholesterol (Davignon et al., “HMG-CoA reductase inhibitors: a look back and a look ahead,” Can. J. Cardiol. 8:843-64 (1992)). Bile-acid-binding resins sequester bile acids in the intestine, thereby interrupting the enterohepatic circulation of bile acids and increasing the elimination of cholesterol from the body. These are effective therapies for some patients with hyperlipidemia; however, adverse effects have been observed in up to 30% of the patients, suggesting the need for alternative therapies. Mutations in the gene encoding the LDL-receptor or apolipoprotein B can cause a human genetic disease known as familial hypercholesterolemia, characterized by an elevated level of cholesterol and early atherosclerosis due to the defect in LDL-receptor mediated cholesterol uptake by cells (Goldstein et al., Familial hypercholesterolemia,” In The Metabolic and Molecular Bases of Inherited Disease, Vol. 2., p1981-2030, Scriver et al. (eds.), McGraw-Hill, New York (1995)). Therapy for children with this disorder is needed in order to prevent morbidity or mortality, however the National Cholesterol Education Program (NCEP) recommends consideration of drug treatment only for children 10 years of age or older due to the risk that prolonged drug therapy may impair growth and pubertal development. Developing alternative approaches for lowering serum LDL levels is therefore essential for the sectors of the population still at risk.

Stimulating hepatic apolipoprotein B mRNA editing is a means of reducing serum LDL through the reduction in synthesis and secretion of apolipoprotein B100 containing VLDL. In most mammals (including humans), apolipoprotein B MRNA editing is carried out only in the small intestine. The presence of substantial editing in liver (found in 4 species) is associated with a less atherogenic lipoprotein profile compared with animals that do not have liver editing activity (Greeve et al., “Apolipoprotein B mRNA editing in 12 different mammalian species: hepatic expression is reflected in low concentrations of apoB-containing plasma lipoproteins,” J. Lipid Res. 34:1367-1383 (1993)). APOBEC-1 is expressed in all tissues that carry out apolipoprotein B mRNA editing (Teng et al., “Molecular cloning of an apo B messenger RNA editing protein,” Science 260:18116-1819 (1993)). Human liver does not express APOBEC-1 but it does express sufficient auxiliary proteins to complement exogenous APOBEC-1 in apolipoprotein B MRNA editing in transfected cells (Teng et al., “Molecular cloning of an apo B messenger RNA editing protein,” Science 260:18116-1819 (1993); Sowden et al., “Apolipoprotein B RNA Sequence 3′ of the mooring sequence and cellular sources of auxiliary factors determine the location and extent of promiscuous editing,” Nucleic Acids Res. 26:1644-1652 (1998)).

Transgenic experiments aiming to enhance hepatic editing through apobec-1 gene transfer have shown a marked lowering of plasma apolipoprotein B 100 and significant reduction of serum LDL (Teng et al., “Adenovirus-mediated gene transfer of rat apolipoprotein B mRNA editing protein in mice virtually eliminates apolipoprotein B100 and normal low density lipoprotein production,” J. Biol. Chem. 269:29395-29404 (1994); Hughs et al., “Gene transfer of cytidine deaminase APOBEC-1 lowers lipoprotein(a) in transgenic nice and induces apolipoprotein B mRNA editing in rabbits,“ Hum. Gene Ther. 7:39-49 (1996); Nakamuta et al., “Complete phenotypic characterization of the apobec-1 knockout mice with a wild-type genetic background and a human apolipoprotein B transgenic background, and restoration of apolipoprotein B mRNA editing by somatic gene transfer of Apobec-1,” J. Biol. Chem. 271:25981-25988 (1996); Kozarsky et al., “Hepatic expression of the catalytic subunit of the apolipoprotein B mRNA editing enzyme ameliorates hypercholesterolemia in LDL receptor-deficient rabbits,” Hum. Gene Ther. 7:943-957 (1996); Farese et al., “Phenotypic analysis of mice expressing exclusively apolipoprotein B48 or apolipoprotein B100 ,” Proc. Natl. Acad. Sci. USA 93:6393-6398 (1996); Qian et al., “Low expression of the apolipoprotein B mRNA editing transgene in mice reduces LDL but does not cause liver dysplasia or tumors,” Arteriosc. Thromb. Vasc. Biol. 18:1013-1020 (1998); Wu et al., “Normal perinatal rise in serum cholesterol is inhibited by hepatic delivery of adenoviral vector expressing apolipoprotein B mRNA editing enzyme in rabbits,” J. Surg. Res. 85:148-157 (1999)). Apolipoprotein B100 is not essential for life as mice that synthesize exclusively apolipoprotein B48 (apolipoprotein B48-only mice) generated through targeted mutagenesis developed normally, were healthy and fertile. Compared with wild-type mice fed on a chow diet, the level of LDL-cholesterol was lower in apolipoprotein B48-only mice (Farese et al., “Phenotypic analysis of mice expressing exclusively apolipoprotein B48 or apolipoprotein B100,” Proc. Natl. Acad. Sci. USA 93:6393-6398 6398 (1996)). However, the induction of apolipoprotein B mRNA editing activity through apobec-1 gene transfer and tissue-specific overexpression poses a significant challenge in that it has induced hepatocellular dysplasia and carcinoma in transgenic mice and rabbits (Yamanaka et al., “Apolipoprotein B mRNA editing protein induces hepatocellular carcinoma and dysplasia in transgenic animals.,” Proc. Natl. Acad. Sci. USA 92: 8483-8487 (1995); Yamanaka et al., “Hyperediting of multiple cytidines of apolipoprotein B mRNA by APOBEC-1 requires auxiliary protein(s) but not a mooring sequence motif,” J. Biol. Chem. 271:11506-11510 (1996); Yamanaka et al., “A novel translational repressor mRNA is edited extensively in livers containing tumors caused by the transgene expression of the apoB mRNA editing enzyme,” Genes & Dev. 11:321-333 (1997)). This was proposed to be due to persistent high levels of APOBEC-1 expression resulting in unregulated and nonspecific mRNA editing (Sowden et al., “Overexpression of APOBEC-1 results in mooring-sequence-dependent promiscuous RNA editing,” J. Biol. Chem 271:3011-3017 (1996); Yamanaka et al., “A novel translational repressor mRNA is edited extensively in livers containing tumors caused by the transgene expression of the apoB mRNA editing enzyme,” Genes & Dev. 11:321-333 (1997); Sowden et al., “Apolipoprotein B RNA Sequence 3′ of the mooring sequence and cellular sources of auxiliary factors determine the location and extent of promiscuous editing,” Nucleic Acids Res. 26:1644-1652 (1998)). Adverse effects were not observed in transgenic animals with low to moderate levels of APOBEC-1 expression (Teng et al., “Adenovirus-mediated gene transfer of rat apolipoprotein B mRNA editing protein in mice virtual eliminates apolipoprotein B100and normal low density lipoprotein production,” J. Biol. Chem. 269:29395-29404 (1994); Qian et al., “Low expression of the apolipoprotein B mRNA editing transgene in mice reduces LDL but does not cause liver dysplasia or tumors,” Arteriosc. Thromb. Vasc. Biol. 18:1013-1020 (1998); Wu et al., “Normal perinatal rise in serum cholesterol is inhibited by hepatic delivery of adenoviral vector expressing apolipoprotein B mRNA editing enzyme in rabbits,” J. Surg. Res. 85:148-157 (1999)). Despite the limited success of apobec-1 gene therapy in modifying apolipoprotein B mRNA editing, such gene therapy poses too great a risk of adverse effects stemming from either persistent elevated levels of APOBEC-1 expression or problems associated with the use of infective transformation vectors (e.g., adenoviral vectors).

For these reasons, it would be desirable to identify an approach to achieve apolipoprotein B mRNA editing, where its induction can be maintained at low levels and importantly, achieved in a transient manner. Moreover, it would be desirable to identify an approach to achieve apolipoprotein B mRNA editing which is substantially free of the side-effects observed with reported gene therapy approaches. The present invention is directed to overcoming these and other deficiencies in the art.

### SUMMARY OF THE INVENTION

A first aspect of the present invention relates to a chimeric protein including: a first polypeptide that includes a protein transduction domain and a second polypeptide that includes APOBEC-1 or a fragment thereof which can edit mRNA encoding apolipoprotein B.

A second aspect of the present invention relates to a chimeric protein including: a first polypeptide that includes a protein transduction domain; and a second polypeptide that includes APOBEC-1 Complementation Factor (“ACF”) or a fragment thereof which can bind to apolipoprotein B mRNA to facilitate editing of the mRNA by APOBEC-1.

Third and fourth aspects of the present invention relate to DNA molecules which encode one of the chimeric proteins of the present invention. DNA constructs, expression vectors, and recombinant host cells including such DNA molecules are also disclosed.

A fifth aspect of the present invention relates to a composition which includes: a pharmaceutically acceptable carrier and a chimeric protein of the present invention.

A sixth aspect of the present invention relates to a composition which includes: a first chimeric protein including a first polypeptide that includes a protein transduction domain and a second polypeptide that includes APOBEC-1 or a fragment thereof which can edit mRNA encoding apolipoprotein B; and a second chimeric protein including a first polypeptide that includes a protein transduction domain and a second polypeptide that includes ACF or a fragment thereof which can bind to apolipoprotein B mRNA to facilitate editing of the mRNA by APOBEC-1or the fragment thereof.

A seventh aspect of the present invention relates to a delivery device which includes either a chimeric protein of the present invention or a composition of the present invention.

An eighth aspect of the present invention relates to a method of modifying apolipoprotein B mRNA editing in vivo which includes: contacting apolipoprotein B mRNA in a cell with a chimeric protein including a first polypeptide that includes a protein transduction domain and a second polypeptide that includes APOBEC-1 or a fragment thereof which can edit mRNA encoding apolipoprotein B, under conditions effective to increase the concentration of apolipoprotein B48 which is secreted by the cell as compared to the concentration of apolipoprotein B 100 which is secreted by the cell, relative to an untreated cell.

A ninth aspect of the present invention relates to a method of reducing serum LDL levels which includes: delivering into one or more cells of a patient, without genetically modifying the cells, an amount of a protein comprising APOBEC-1 or a fragment thereof which can edit mRNA encoding apolipoprotein B, which amount is effective to increase the concentration of VLDL-apolipoprotein B48 that is secreted by the one or more cells into serum and, consequently, reduce the serum concentration of LDL.

A tenth aspect of the present invention relates to a method of treating or preventing an atherogenic disease or disorder which includes: administering to a patient an effective amount of a protein including APOBEC-1 or a fragment thereof which can edit mRNA encoding apolipoprotein B, wherein upon said administering the protein is taken up by one or more cells of the patient that can synthesize and secrete VLDL-apolipoprotein B under conditions which are effective to increase the concentration of VLDL-apolipoprotein B48 that is secreted by the one or more cells into serum, whereby rapid clearing of VLDL-apolipoprotein B48 from serum decreases the serum concentration of LDL to treat or prevent the atherogenic disease or disorder.

An eleventh aspect of the present invention relates to a liposome or noisome which is targeted for uptake by a liver cell, the liposome or niosome containing (i) APOBEC-1 or a fragment thereof which is effective to edit apolipoprotein B mRNA, (ii) ACF or a fragment thereof which is effective to bind apolipoprotein B mRNA, or (iii) a combination thereof. Compositions which include the liposome or niosome are also disclosed.

The present invention demonstrates the efficacy of protein-mediated delivery to increase intracellular APOBEC-1 in cells which produce and secrete VLDL-apolipoprotein B. By increasing the extent of apolipoprotein B mRNA editing in vivo, it is possible to modify the ratio of VLDL-apolipoprotein B48 to VLDL-apolipoprotein B 100 which is secreted by such cells, specifically increasing the relative serum concentration of VLDL-apolipoprotein B48 and decreasing the relative serum concentration of VLDL-apolipoprotein B 100. Due to the nature of these complexes, the B48 complex is cleared much more rapidly from serum, minimizing the conversion of VLDL into LDL, a major atherogenic disease factor. By minimizing the amount of VLDL-apolipoprotein B 100 and increasing the amount of VLDL-apolipoprotein B48, it is possible to both treat and prevent atherogenic diseases or disorders. Moreover, by using protein delivery, it is possible to avoid the apparently unavoidable side effects of gene therapy. These results presented here open new possibilities for the treatment of hyperlipidemia through the induction of precisely controlled hepatic editing activity.

### BRIEF DESCRIPTION OF THE DRAWINGS

FIGS. 1A-D illustrate the structure (1A) and both nucleotide (1B-C, SEQ ID No: 1) and amino acid (1D, SEQ ID No: 2) sequences for an exemplary first chimeric protein (designated TAT-hAPOBEC-CMPK) specific for human apolipoprotein B mRNA editing. In FIG. 1B-C, the region encoding human APOBEC-1 is shown in lowercase letters and the start codon for this construct is at the beginning of the sequence. The sequences encoding a TAT protein transduction domain and a hemagglutinin domain are shown in uppercase letters near the 5′ end (i.e., upstream of the APOBEC-1 sequence). The sequence encoding CMPK is shown 3′ of the APOBEC-1 sequence in uppercase letters. At the 3′ terminal region and shown in lowercase letters is a sequence encoding a histidine tag. In FIG. 1D, beginning from the N-terminal end, the TAT protein transduction domain is shown in bold, followed by the hemagglutinin domain also shown in bold, human APOBEC-1 shown underlined, CMPK also shown underlined, and the histidine tag shown in bold at the C-terminus.

FIGS. 2A-D illustrate the structure (2A) and both nucleotide (2B-C, SEQ ID No: 3) and amino acid (2D, SEQ ID No: 4) sequences for an exemplary first chimeric protein (designated TAT-rAPOBEC-CMPK) specific for rat apolipoprotein B mRNA editing. In FIGS. 2B-C, the region encoding rat APOBEC-1 is shown in lowercase letters and the start codon for this construct is at the beginning of the sequence. The sequences encoding a TAT protein transduction domain and a hemagglutinin domain are shown in uppercase letters near the 5′ end (i.e., upstream of the APOBEC-1 sequence). The sequence encoding CMPK is shown 3′ of the APOBEC-1 sequence in uppercase letters. At the 3′ terminal region and shown in lowercase letters is a sequence encoding a histidine tag. In FIG. 2D, beginning from the N-terminal end, the TAT protein transduction domain is shown in bold, followed by the hemagglutinin domain also shown in bold, rat APOBEC-1 shown underlined, CMPK also shown underlined, and the histidine tag shown in bold at the C-terminus.

FIGS. 3A-C illustrate the structure (3A) and both nucleotide (3B, SEQ ID No: 5) and amino acid (3C, SEQ ID No: 6) sequences for an exemplary second chimeric protein (designated TAT-hACF) specific for complementing human APOBEC-1. In FIG. 3B, the region encoding human ACF is shown in lowercase letters and the start codon for this construct is at the beginning of the sequence. The sequence encoding a TAT protein transduction domain and a hemagglutinin domain is shown in uppercase letters near the 5′ end (i.e., upstream of the ACF sequence). At the 3′ terminal region and shown in lowercase letters is a sequence encoding a histidine tag. In FIG. 3C, beginning from the N-terminal end, the TAT protein transduction domain is shown in bold, followed by the hemagglutinin domain also shown in bold, human ACF shown underlined, and the histidine tag shown in bold at the C-terminus.

FIGS. 4A-C illustrate the structure (4A) and both nucleotide (4B, SEQ ID No: 7) and amino acid (4C, SEQ ID No: 8) sequences for an exemplary second chimeric protein (designated TAT-rACF) specific for complementing rat APOBEC-1. In FIG. 4B, the region encoding rat ACF is shown in lowercase letters and the start codon for this construct is at the beginning of the sequence. The sequence encoding a TAT protein transduction domain and a hemagglutinin domain is shown in uppercase letters near the 5′ end (i.e., upstream of the ACF sequence). At the 3′ terminal region and shown in lowercase letters is a sequence encoding a histidine tag. In FIG. 4C, beginning from the N-terminal end, the TAT protein transduction domain is shown in bold, followed by the hemagglutinin domain also shown in bold, rat ACF shown underlined, and the histidine tag shown in bold at the C-terminus.

FIGS. 5A-B illustrate the purification of full-length TAT-rAPOBEC-CMPK protein. In FIG. 5A, a schematic image illustrates generally the structure of a prokaryotic expression vector, pET-24b, encoding the TAT fusion protein. FIG. 5B illustrates the image of a gel following two-column purification and silver-staining. The TAT fusion protein is the only protein recovered in significant concentrations.

FIGS. 6A-F are images of immuno-stained cells exposed to the TAT fusion protein TAT-rAPOBEC-CMPK. McArdle cells were treated with 650 nM of recombinant TAT-rAPOBEC-CMPK for the indicated times (1 h, 6 h, or 24 h). Cells were fixed, permeabilized, reacted with antibody to the HA epitope and FITC-conjugated anti-mouse secondary antibody and mounted in DAPI containing buffer as described in the Examples. Arrowheads indicated the position of select nuclei.

FIGS. 7A-F are images of immuno-stained cell exposed to TAT-CMPK fusion protein. McArdle cells were treated with 1125 nM of recombinant TAT-CMPK for the indicated times (1 h, 6 h, or 24 h). Cells were fixed, permeabilized, reacted with antibody to the HA epitope and FITC-conjugated anti-mouse secondary antibody and mounted in DAPI containing buffer as described in the Examples. Arrowheads indicated the position of select nuclei.

FIG. 8 is an image of a gel indicating that TAT-CMPK did not stimulate editing. McArdle cells were treated with 45 nM, 225 nM and 1125 nM of recombinant TAT-CMPK for 24 h. Total cellular RNA was isolated and apolipoprotein B mRNA was selectively amplified by reverse transcription-polymerase chain reaction (“RT-PCR”) and the proportion of edited apolipoprotein B RNA determined by poisoned primer extension as described in the Examples. CAA, primer extension product corresponding to unedited RNA; UAA, primer extension product corresponding to edited RNA; P, primer.

FIG. 9 is an image of a gel indicating that TAT-rAPOBEC-CMPK increased editing activity in McArdle cells. The TAT fusion protein (360 nM or 62 μg protein/ml media) was added into cell culture media and RNAs were isolated subsequent to treatment from wild type McArdle cells at the indicated time points. Control cells were treated with a corresponding aliquot of buffer B used to dialyze the recombinant protein. The editing efficiency was calculated as described in the Examples. The standard deviations for each of the lanes on the gel, reading left to right, are as follows: 0.9, 2.2, 3.8, 2.1, 1.1, 0.9, 0.2, n=3. CAA, primer extension product corresponding to unedited RNA; UAA, primer extension product corresponding to edited RNA; P, primer.

FIG. 10 is an image of a gel indicating that TAT fusion protein increased editing activity in primary rat hepatocytes. Hepatocytes were prepared and treated with TAT-rAPOBEC-CMPK as described in the Examples. Control cells were treated with a corresponding aliquot of buffer B used to dialyze the recombinant protein. The increase in editing activity caused by TAT fusion protein was apparent. The standard deviations for each of the lanes on the gel, reading left to right, are as follows: 2.2, 3.6, 2.5, 1.9, n=3.

FIG. 11 is an image showing the changes in secreted lipoprotein profile due to TAT-rAPOBEC-CMPK treatment. Primary hepatocytes were treated with TAT fusion protein first, then labeled with [³⁵S]methionine and [³⁵S]cysteine. Control cells (−) were treated with a corresponding aliquot of buffer B used to dialyze the recombinant protein. Cell culture media were collected, apolipoprotein B48 and apolipoprotein B 100 were precipitated by anti-apoB antibody and separated by SDS-PAGE. The second band below apolipoprotein B48 might have been due to protein degradation and the band between apolipoprotein B 100 and apolipoprotein B48 could be C-3 complement. The editing efficiency of the same cells is shown at the bottom

The results are from a single experiment representative three experiments with similar results.

### DETAILED DESCRIPTION OF THE INVENTION

The present invention relates to protein-mediated approaches for regulating apolipoprotein B mRNA editing and, therefore, regulating the relative concentration of secreted apolipoprotein B derivatives, which offers an approach for controlling the serum levels of atherogenic disease factors such as low density lipoproteins (“LDL”) which associates with apolipoprotein B and its derivatives.

According to one aspect of the present invention, a first chimeric protein is provided for such uses. The first chimeric protein includes a first polypeptide that includes a protein transduction domain and a second polypeptide that includes APOBEC-1 or a fragment thereof which can edit mRNA encoding apolipoprotein B.

The first polypeptide can be any protein, or polypeptide fragment thereof, which is suitable for inducing cellular uptake of the chimeric protein.

By way of example, protein transduction domains from several known proteins can be employed, including without limitation, HIV-1 Tat protein, Drosophila homeotic transcription factor (ANTP), and HSV-1 VP22 transcription factor (Schwarze et al., “In vivo protein transduction: Intracellular delivery of biologically active proteins, compounds, and DNA,” TiPS 21:45-48 (2000), which is hereby incorporated by reference in its entirety).

A preferred protein transduction domain is the protein transduction domain of the human immunodeficiency virus (“HIV”) tat protein. An exemplary HIV tat protein transduction domain has an amino acid sequence of SEQ ID No: 9 as follows:

Arg Lys Lys Arg Arg Gln Arg Arg Arg
5

This protein transduction domain has also been noted to be a nuclear translocation domain (HIV Sequence Compendium 2000, Kuiken et al. (eds.), Theoretical Biology and Biophysics Group, Los Alamos National Laboratory, which is hereby incorporated by reference in its entirety). One DNA molecule which encodes the HIV tat protein transduction domain has a nucleotide sequence of SEQ ID No: 10 as follows:

agaaaaaaaa gaagacaaag aagaaga 27

Variations of these tat sequences can also be employed. Such sequence variants have been reported in HIV Sequence Compendium 2000, Kuiken et al. (eds.), Theoretical Biology and Biophysics Group, Los Alamos National Laboratory, which is hereby incorporated by reference in its entirety.

Other cellular uptake polypeptides and their use have been described in the literature, including membrane-permeable sequences of the SN50 peptide, the Grb2 SH2 domain, and integrin β₃, β₁, and α_{IIb} cytoplasmic domains (Hawiger, “Noninvasive intracellular delivery of functional peptides and proteins,” Curr. Opin. Chem. Biol. 3:89-94 (1999), which is hereby incorporated by reference in its entirety).

The second polypeptide can be either a full length APOBEC-1 or a fragment thereof which includes the catalytic domain thereof. The APOBEC-1 protein or fragment thereof is a mammalian APOBEC-1 protein or fragment thereof, including without limitation, human, rat, mouse, etc.

The full length human APOBEC-1 has an amino acid sequence according to SEQ ID No: 11 as follows:

```
Met Thr Ser Glu Lys Gly Pro Ser Thr Gly Asp Pro Thr Leu Arg Arg
    1 5 10 15
Arg Ile Glu Pro Trp Glu Phe Asp Val Phe Tyr Asp Pro Arg Glu Leu
                          20 25 30
Arg Lys Glu Ala Cys Leu Leu Tyr Glu Ile Lys Trp Gly Met Ser Arg
                  35 40 45
Lys Ile Trp Arg Ser Ser Gly Lys Asn Thr Thr Asn His Val Glu Val
          50 55 60
Asn Phe Ile Lys Lys Phe Thr Ser Glu Arg Asp Phe His Pro Ser Ile
  65 70 75 80
Ser Cys Ser Ile Thr Trp Phe Leu Ser Trp Ser Pro Cys Trp Glu Cys
                                  85 90 95
Ser Gln Ala Ile Arg Glu Phe Leu Ser Arg His Pro Gly Val Thr Leu
                        100 105 110
Val Ile Tyr Val Ala Arg Leu Phe Trp His Met Asp Gln Gln Asn Arg
                115 120 125
Gln Gly Leu Arg Asp Leu Val Asn Ser Gly Val Thr Ile Gln Ile Met
        130 135 140
Arg Ala Ser Glu Tyr Tyr His Cys Trp Arg Asn Phe Val Asn Tyr Pro
145 150 155 160
Pro Gly Asp Glu Ala His Trp Pro Gln Tyr Pro Pro Leu Trp Met Met
                                165 170 175
Leu Tyr Ala Leu Glu Leu His Cys Ile Ile Leu Ser Leu Pro Pro Cys
                        180 185 190
Leu Lys Ile Ser Arg Arg Trp Gln Asn His Leu Thr Phe Phe Arg Leu
                195 200 205
His Leu Gln Asn Cys His Tyr Gln Thr Ile Pro Pro His Ile Leu Leu
        210 215 220
Ala Thr Gly Leu Ile His Pro Ser Val Ala Trp Arg
225 230 235
```

This human APOBEC-1 sequence is reported at Genbank Accession No. NP₁₃001635, which is hereby incorporated by reference in its entirety. The full length human APOBEC-1 is believed to include a putative bipartite nuclear localization signal between amino acid residues 15-34, a catalytic center between amino acid residues 61-98, and a putative cytoplasmic retention signal between amino acid residues 173-229. A cDNA sequence which encodes the full length human APOBEC-1 is set forth as SEQ ID No: 12 as follows:

| | |
|---|---|
| atgacttctg agaaaggtcc ttcaaccggt gaccccactc tgaggagaag aatcgaaccc | 60 |
| tgggagtttg acgtcttcta tgaccccaga gaacttcgta aagaggcctg tctgctctac | 120 |
| gaaatcaagt ggggcatgag ccggaagatc tggcgaagct caggcaaaaa caccaccaat | 180 |
| cacgtggaag ttaattttat aaaaaaattt acgtcagaaa gagattttca cccatccatc | 240 |
| agctgctcca tcacctggtt cttgtcctgg agtccctgct gggaatgctc ccaggctatt | 300 |
| agagagtttc tgagtcggca ccctggtgtg actctagtga tctacgtagc tcggcttttt | 360 |
| tggcacatgg atcaacaaaa tcggcaaggt ctcagggacc ttgttaacag tggagtaact | 420 |
| attcagatta tgagagcatc agagtattat cactgctgga ggaattttgt caactaccca | 480 |
| cctggggatg aagctcactg gccacaatac ccacctctgt ggatgatgtt gtacgcactg | 540 |
| gagctgcact gcataattct aagtcttcca ccctgtttaa agatttcaag aagatggcaa | 600 |
| aatcatctta catttttcag acttcatctt caaaactgcc attaccaaac gattccgcca | 660 |
| cacatccttt tagctacagg gctgatacat ccttctgtgg cttggagatg a 711 | |

The full length rat APOBEC-1 has an amino acid sequence according to SEQ ID No: 13 as follows:

```
Met Ser Ser Glu Thr Gly Pro Val Ala Val Asp Pro Thr Leu Arg Arg
    1 5 10 15
Arg Ile Glu Pro His Glu Phe Glu Val Phe Phe Asp Pro Arg Glu Leu
                          20 25 30
Arg Lys Glu Thr Cys Leu Leu Tyr Glu Ile Asn Trp Gly Gly Arg His
                  35 40 45
Ser Ile Trp Arg His Thr Ser Gln Asn Thr Asn Lys His Val Glu Val
          50 55 60
Asn Phe Ile Glu Lys Phe Thr Thr Glu Arg Tyr Phe Cys Pro Asn Thr
  65 70 75 80
Arg Cys Ser Ile Thr Trp Phe Leu Ser Trp Ser Pro Cys Gly Glu Cys
                                  85 90 95
Ser Arg Ala Ile Thr Glu Phe Leu Ser Arg Tyr Pro His Val Thr Leu
                        100 105 110
Phe Ile Tyr Ile Ala Arg Leu Tyr His His Ala Asp Pro Arg Asn Arg
                115 120 125
Gln Gly Leu Arg Asp Leu Ile Ser Ser Gly Val Thr Ile Gln Ile Met
        130 135 140
Thr Glu Gln Glu Ser Gly Tyr Cys Trp Arg Asn Phe Val Asn Tyr Ser
145 150 155 160
Pro Ser Asn Glu Ala His Trp Pro Arg Tyr Pro His Leu Trp Val Arg
                                165 170 175
Leu Tyr Val Leu Glu Leu Tyr Cys Ile Ile Leu Gly Leu Pro Pro Cys
                        180 185 190
Leu Asn Ile Leu Arg Arg Lys Gln Pro Gln Leu Thr Phe Phe Thr Ile
                195 200 205
Ala Leu Gln Ser Cys His Tyr Gln Arg Leu Pro Pro His Ile Leu Trp
        210 215 220
Ala Thr Gly Leu Lys
225
```

This rat APOBEC-1 sequence is reported at Genbank Accession No. P38483, which is hereby incorporated by reference in its entirety. Recombinant studies using rat APOBEC-1 have demonstrated that an N-terminal region, containing the putative nuclear localization signal, is required for nuclear distribution of APOBEC-1 while a C-terminal region, containing a putative cytoplasmic retention signal (Yang et al., “Multiple protein domains determine the cell type-specific nuclear distribution of the catalytic subunit required for apolipoprotein B mRNA editing,” Proc. Natl. Acad. Sci. USA 94:13075-13080 (1997), which is hereby incorporated by reference in its entirety. A cDNA sequence which encodes the full length rat APOBEC-1 is set forth as SEQ ID No: 14 as follows:

| | |
|---|---|
| atgagttccg asacaggccc tgtagctgtt gatcccactc tgaggagaag aattgagccc | 60 |
| cacgagtttg aagtcttctt tgacccccgg gaacttcgga aagagacctg tctgctgtat | 120 |
| gagatcaact gg9gaggaag gcacagcatc tggcgacaca cgagccaaaa caccaacaaa | 180 |
| cacgttgaag tcaatttcat agaaaaattt actacagaaa gatacttttg tccaaacacc | 240 |
| agatgctcca ttacctggtt cctgtcctgg agtccctgtg gggagtgctc cagggccatt | 300 |
| acagaatttt tgagccgata cccccatgta actctgttta tttatatagc acggctttat | 360 |
| caccacgcag atcctcgaaa tcggcaagga ctcagggacc ttattagcag cggtgttact | 420 |
| atccagatca tgacggagca agagtctggc tactgctgga ggaattttgt caactactcc | 480 |
| ccttcgaatg aagctcattg gccaaggtac ccccatctgt gggtgaggct gtacgtactg | 540 |
| gaactctact gcatcatttt aggacttcca ccctgtttaa atattttaag aagaaaacaa | 600 |
| cctcaactca cgtttttcac gattgctctt caaagctgcc attaccaaag gctaccaccc | 660 |
| cacatcctgt gggccacagg gttgaaatga 690 | |

The cDNA molecule is reported at Genbank Accession No. L07114, which is hereby incorporated by reference in its entirety.

The full length mouse APOBEC-1 has an amino acid sequence according to SEQ ID No: 15 as follows:

```
Met Ser Ser Glu Thr Gly Pro Val Ala Val Asp Pro Thr Leu Arg Arg
    1 5 10 15
Arg Ile Glu Pro His Glu Phe Glu Val Phe Phe Asp Pro Arg Glu Leu
                          20 25 30
Arg Lys Glu Thr Cys Leu Leu Tyr Glu Ile Asn Trp Gly Gly Arg His
                  35 40 45
Ser Val Trp Arg His Thr Ser Gln Asn Thr Ser Asn His Val Glu Val
          50 55 60
Asn Phe Leu Glu Lys Phe Thr Thr Glu Arg Tyr Phe Arg Pro Asn Thr
  65 70 75 80
Arg Cys Ser Ile Thr Trp Phe Leu Ser Trp Ser Pro Cys Gly Glu Cys
                                          85 90 95
Ser Arg Ala Ile Thr Glu Phe Leu Ser Arg His Pro Tyr Val Thr Leu
                        100 105 110
Phe Ile Tyr Ile Ala Arg Leu Tyr His His Thr Asp Gln Arg Asn Arg
                115 120 125
Gln Gly Leu Arg Asp Leu Ile Ser Ser Gly Val Thr Ile Gln Ile Met
        130 135 140
Thr Glu Gln Glu Tyr Cys Tyr Cys Trp Arg Asn Phe Val Asn Tyr Pro
145 150 155 160
Pro Ser Asn Glu Ala Tyr Trp Pro Arg Tyr Pro His Leu Trp Val Lys
                                165 170 175
Leu Tyr Val Leu Glu Leu Tyr Cys Ile Ile Leu Gly Leu Pro Pro Cys
                        180 185 190
Leu Lys Ile Leu Arg Arg Lys Gln Pro Gln Leu Thr Phe Phe Thr Ile
                195 200 205
Thr Leu Gln Thr Cys His Tyr Gln Arg Ile Pro Pro His Leu Leu Trp
        210 215 220
Ala Thr Gly Leu Lys
225
```

This mouse APOBEC-1 sequence is reported at Genbank Accession No. NP₁₃112436, which is hereby incorporated by reference in its entirety. A cDNA sequence which encodes the full length mouse APOBEC-1 is set forth as SEQ ID No: 16 as follows:

| | |
|---|---|
| atgagttccg agacaggccc tgtagctgtt gatcccactc tgaggagaag aattgagccc | 60 |
| cacgagtttg aagtcttctt tgacccccgg gagcttcgga aagagacctg tctgctgtat | 120 |
| gagatcaact ggggtggaag gcacagtgtc tggcgacaca cgagccaaaa caccagcaac | 180 |
| cacgttgaag tcaacttctt agaaaaattt actacagaaa gatactttcg tccgaacacc | 240 |
| agatgctcca ttacctggtt cctgtcctgg agtccctgcg gggagtgctc cagggccatt | 300 |
| acagagtttc tgagccgaca cccctatgt& actctgttta tttacatagc acggctttat | 360 |
| caccacacgg atcagcgaaa ccgccaagga ctcagggacc ttattagcag cggtgtgact | 420 |
| atccagatca tgacagagca agagtattgt tactgctgga ggaatttcgt caactacccc | 480 |
| ccttcaaacg aagcttattg gccaaggtac ccccatctgt gggtgaaact gtatgtattg | 540 |
| gagctctact gcatcatttt aggacttcca ccctgtttaa aaattttaag aagaaagcaa | 600 |
| cctcaactca cgtttttcac aattactctt caaacctgcc attaccaaag gataccaccc | 660 |
| catctccttt gggctacagg gttgaaatga 690 | |

The cDNA molecule is reported at Genbank Accession No. NM₁₃031159, which is hereby incorporated by reference in its entirety.

The first chimeric protein of the present invention can also include one or more other polypeptide sequences, including without limitation: (i) a polypeptide that includes a cytoplasmic localization protein or a fragment thereof which, upon cellular uptake of the first chimeric protein, localizes the first chimeric protein to the cytoplasm; (ii) a polypeptide that includes a plurality of adjacent histidine residues; and (iii) a polypeptide that includes an epitope tag.

The polypeptide that includes a cytoplasmic localization protein or a fragment thereof can be any protein, or fragment thereof, which can effectively retain the first chimeric protein within the cytoplasm of a cell into which the first chimeric protein has been translocated. One such protein is chicken muscle pyruvate kinase (“CMPK”), which has an amino acid sequence of SEQ ID No: 17 as follows:

```
Met Ser Lys His His Asp Ala Gly Thr Ala Phe Ile Gln Thr Gln Gln
    1 5 10 15
Leu His Ala Ala Met Ala Asp Thr Phe Leu Glu His Met Cys Arg Leu
                          20 25 30
Asp Ile Asp Ser Glu Pro Thr Ile Ala Arg Asn Thr Gly Ile Ile Cys
                  35 40 45
Thr Ile Gly Pro Ala Ser Arg Ser Val Asp Lys Leu Lys Glu Met Ile
          50 55 60
Lys Ser Gly Met Asn Val Ala Arg Leu Asn Phe Ser His Gly Thr His
  65 70 75 80
Glu Tyr His Glu Gly Thr Ile Lys Asn Val Arg Glu Ala Thr Glu Ser
                                  85 90 95
Phe Ala Ser Asp Pro Ile Thr Tyr Arg Pro Val Ala Ile Ala Leu Asp
                        100 105 110
Thr Lys Gly Pro Glu Ile Arg Thr Gly Leu Ile Lys Gly Ser Gly Thr
                115 120 125
Ala Glu Val Glu Leu Lys Lys Gly Ala Ala Leu Lys Val Thr Leu Asp
        130 135 140
Asn Ala Phe Met Glu Asn Cys Asp Glu Asn Val Leu Trp Val Asp Tyr
145 150 155 160
Lys Asn Leu Ile Lys Val Ile Asp Val Gly Ser Lys Ile Tyr Val Asp
                                165 170 175
Asp Gly Leu Ile Ser Leu Leu Val Lys Glu Lys Gly Lys Asp Phe Val
                        180 185 190
Met Thr Glu Val Glu Asn Gly Gly Met Leu Gly Ser Lys Lys Gly Val
                195 200 205
Asn Leu Pro Gly Ala Ala Val Asp Leu Pro Ala Val Ser Glu Lys Asp
        210 215 220
Ile Gln Asp Leu Lys Phe Gly Val Glu Gln Asn Val Asp Met Val Phe
225 230 235 240
Ala Ser Phe Ile Arg Lys Ala Ala Asp Val His Ala Val Arg Lys Val
                                245 250 255
Leu Gly Glu Lys Gly Lys His Ile Lys Ile Ile Ser Lys Ile Glu Asn
                        260 265 270
His Glu Gly Val Arg Arg Phe Asp Glu Ile Met Glu Ala Ser Asp Gly
                275 280 285
Ile Met Val Ala Arg Gly Asp Leu Gly Ile Glu Ile Pro Ala Glu Lys
        290 295 300
Val Phe Leu Ala Gln Lys Met Met Ile Gly Arg Cys Asn Arg Ala Gly
305 310 315 320
Lys Pro Ile Ile Cys Ala Thr Gln Met Leu Glu Ser Met Ile Lys Lys
                                325 330 335
Pro Arg Pro Thr Arg Ala Glu Gly Ser Asp Val Ala Asn Ala Val Leu
                        340 345 350
Asp Gly Ala Asp Cys Ile Met Leu Ser Gly Glu Thr Ala Lys Gly Asp
                355 360 365
Tyr Pro Leu Glu Ala Val Arg Met Gln His Ala Ile Ala Arg Glu Ala
        370 375 380
Glu Ala Ala Met Phe His Arg Gln Gln Phe Glu Glu Ile Leu Arg His
385 390 395 400
Ser Val His His Arg Glu Pro Ala Asp Ala Met Ala Ala Gly Ala Val
                                405 410 415
Glu Ala Ser Phe Lys Cys Leu Ala Ala Ala Leu Ile Val Met Thr Glu
                        420 425 430
Ser Gly Arg Ser Ala His Leu Val Ser Arg Tyr Arg Pro Arg Ala Pro
                435 440 445
Ile Ile Ala Val Thr Arg Asn Asp Gln Thr Ala Arg Gln Ala His Leu
        450 455 460
Tyr Arg Gly Val Phe Pro Val Leu Cys Lys Gln Pro Ala His Asp Ala
465 470 475 480
Trp Ala Glu Asp Val Asp Leu Arg Val Asn Leu Gly Met Asn Val Gly
                                485 490 495
Lys Ala Arg Gly Phe Phe Lys Thr Gly Asp Leu Val Ile Val Leu Thr
                        500 505 510
Gly Trp Arg Pro Gly Ser Gly Tyr Thr Asn Thr Met Arg Val Val Pro
                515 520 525
Val Pro
        530
```

A DNA molecule encoding the full length CMPK has a nucleotide sequence according to SEQ ID No: 18 as follows:

| | |
|---|---|
| atgtcgaagc accacgatgc agggaccgct ttcatccaga cccagcagct gcacgctgcc | 60 |
| atggcagaca cctttctgga gcacatgtgc cgcctggaca tcgactccga gccaaccatt | 120 |
| gccagaaaca ccggcatcat ctgcaccatc ggcccagcct cccgctctgt ggacaagctg | 180 |
| aaggaaatga ttaaatctgg aatgaatgtt gcccgcctca acttctcgca cggcacccac | 240 |
| gagtatcatg agggcacaat taagaacgtg cgagagscca cagagagctt tgcctctgac | 300 |
| ccgatcacct acagacctgt ggctattgca ctggacacca agggacctga aatccgaact | 360 |
| ggactcatca agggaagtgg cacagcagag gtggagctca agaagggcgc agctctcaaa | 420 |
| gtgacgctgg acaatgcctt catggagaac tgcgatgaga atgtgctgtg ggtggactac | 480 |
| aagaacctca tcaaagttat agatgtgggc agcaaaatct atgtggatga cggtctcatt | 540 |
| tccttgctgg ttaaggagaa aggcaaggac tttgtcatga ctgaggttga gaacggtggc | 600 |
| atgcttggta gtaagaaggg agtgaacctc ccaggtgctg cggtcgacct gcctgcagtc | 660 |
| tcagagaagg acattcagga cctgaaattt ggcgtggagc agaatgtgga catggtgttc | 720 |
| gcttccttca tccgcaaagc tgctgatgtc catgctgtca ggaaggtgct aggggaaaag | 780 |
| ggaaagcaca tcaagattat cagcaagatt gagaatcacg agggtgtgcg caggtttgat | 840 |
| gagatcatgs aggccagcga tggcattatg gtggcccgtg gtgacctggg tattgagatc | 900 |
| cctgctgaaa aagtcttcct cgcacagaag atgatgattg ggcgctgcaa cagggctggc | 960 |
| aaacccatca tttgtgccac tcagatgttg gaaagcatga tcaagaaacc tcgcccgacc | 1020 |
| cgcgctgagg gcagtgatgt tgccaatgca gttctggatg gagcagactg catcatgctg | 1080 |
| tctggggaga ccgccaaggg agactaccca ctggaggctg tgcgcatgca gcacgctatt | 1140 |
| gctcgtgagg ctgaggccgc aatgttccat cgtcascagt ttgaagaaat cttacgccac | 1200 |
| agtgtacacc acagggagcc tgctgatgcc atggcagcag gcgcggtgga ggcctccttt | 1260 |
| aagtgcttag cagcagctct gatagttatg accgagtctg gcaggtctgc acacctggtg | 1320 |
| tcccggtacc gcccgcgggc tcccatcatc gccgtcaccc gcaatsacca aacagcacgc | 1380 |
| caggcacacc tgtaccgcgg cgtcttcccc gtgctgtgca agcagccggc ccacgatgcc | 1440 |
| tgggcagagg atgtggatct ccgtgtgaac ctgggcatsa atgtcggcaa agcccgtgga | 1500 |
| ttcttcaaga ccggggacct ggtgatcgtg ctgacgggct ggcgccccgg ctccggctac | 1560 |
| accaacacca tgcgggtggt gcccgtgcca tga 1593 | |

The amino acid sequence and nucleotide sequence for the full length CMPK is reported at Genbank Accession Nos. AAA49021 and J00903, respectively, each of which is hereby incorporated by reference in its entirety.

Fragments of CMPK which afford cytoplasmic retention of the first chimeric protein include, without limitation, polypeptides containing at a minimum residues 1-479 of SEQ ID No: 18.

The polypeptide that includes a plurality of histidine residues preferably contains a sufficient number of histidine residues so as to allow the first chimeric protein containing such histidine residues to be bound by an antibody which recognizes the plurality of histidine residues. One type of DNA molecule encoding Hₙ is (cac)ₙ, where n is greater than 1, but preferably greater than about 5. This His region can be used during immuno-purification, which is described in greater detail below.

The polypeptide that includes an epitope tag can be any epitope tag that is recognized with antibodies raised against the epitope tag. An exemplary epitope tag is a hemagglutinin (“HA”) domain. The HA domain is present only when it is desirable to examine, i.e., in vitro, localization of the first chimeric protein within cells that have translocated it. One suitable HA domain has an amino acid sequence according to SEQ ID No: 19 as follows:

Tyr Pro Tyr Asp Val Pro Asp Tyr Ala
1 5

This HA sequence is encoded by a DNA molecule having a nucleotide sequence according to SEQ ID No: 20 as follows:

tacccctacg acgtgcccga ctacgcc 27

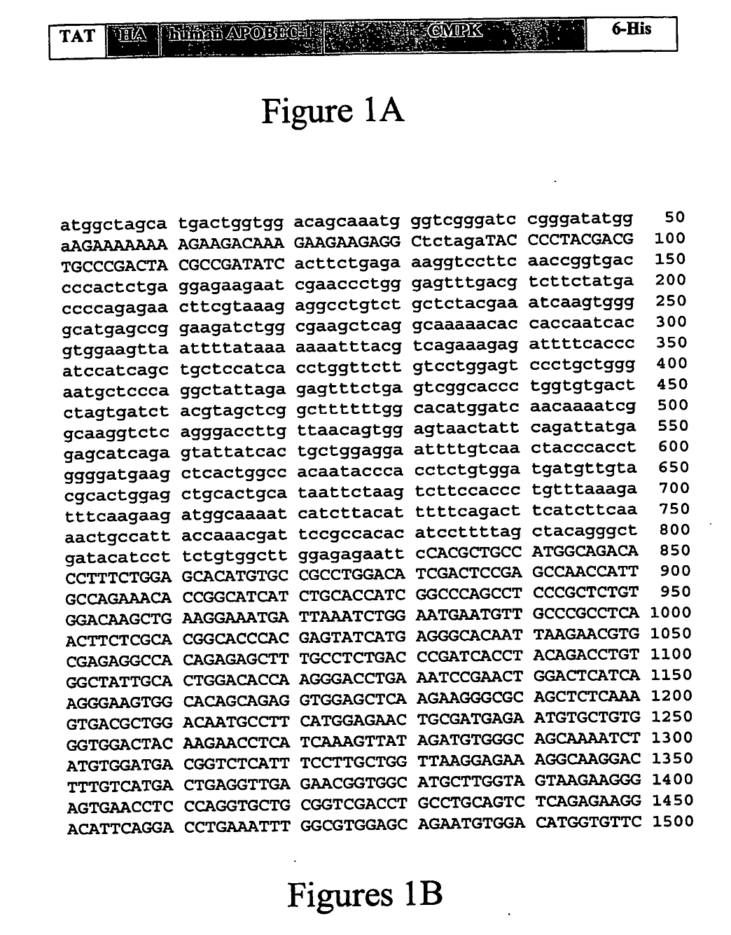

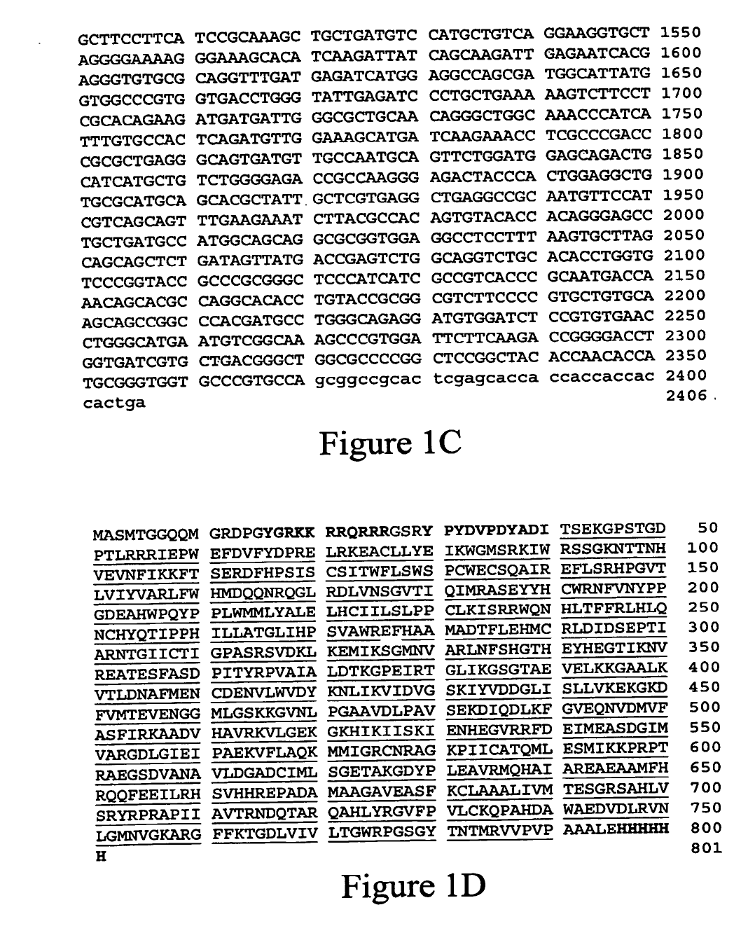

An exemplary first chimeric protein of the present invention which is suitable for use in humans, designated TAT-hAPOBEC-CMPK, is set forth in FIG. 1A. This first chimeric protein (human) includes: an N-terminal HIV tat protein transduction domain, a hemagglutinin domain, a polypeptide fragment of human APOBEC-1, a CMPK domain, and a C-terminal His tag. The amino acid sequence (SEQ ID No: 2) and encoding nucleotide sequence (SEQ ID No: 1) of this exemplary first chimeric protein (human) is set forth in FIGS. 1D and 1B-C, respectively.

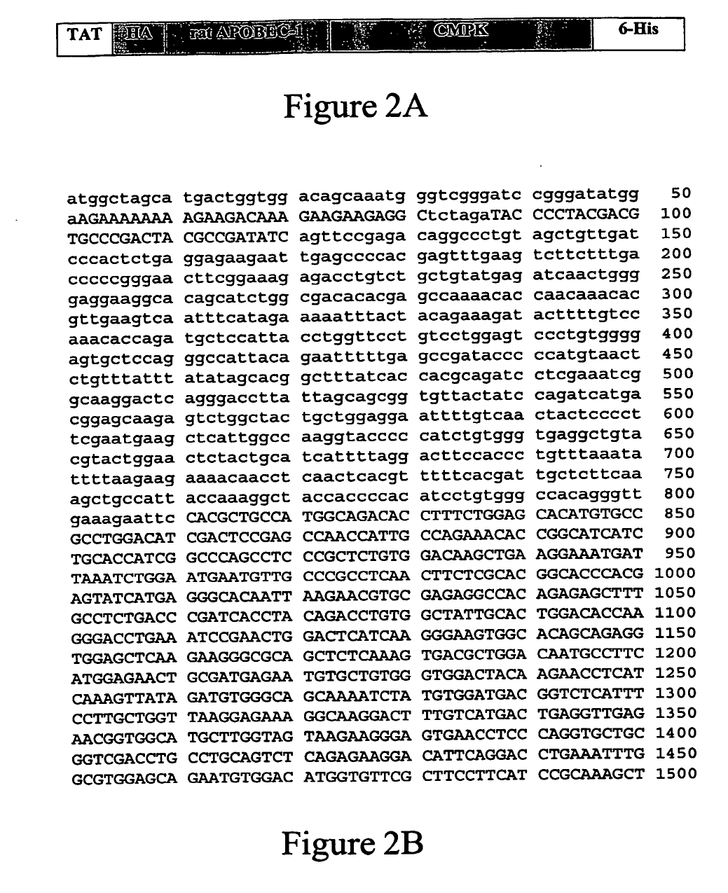

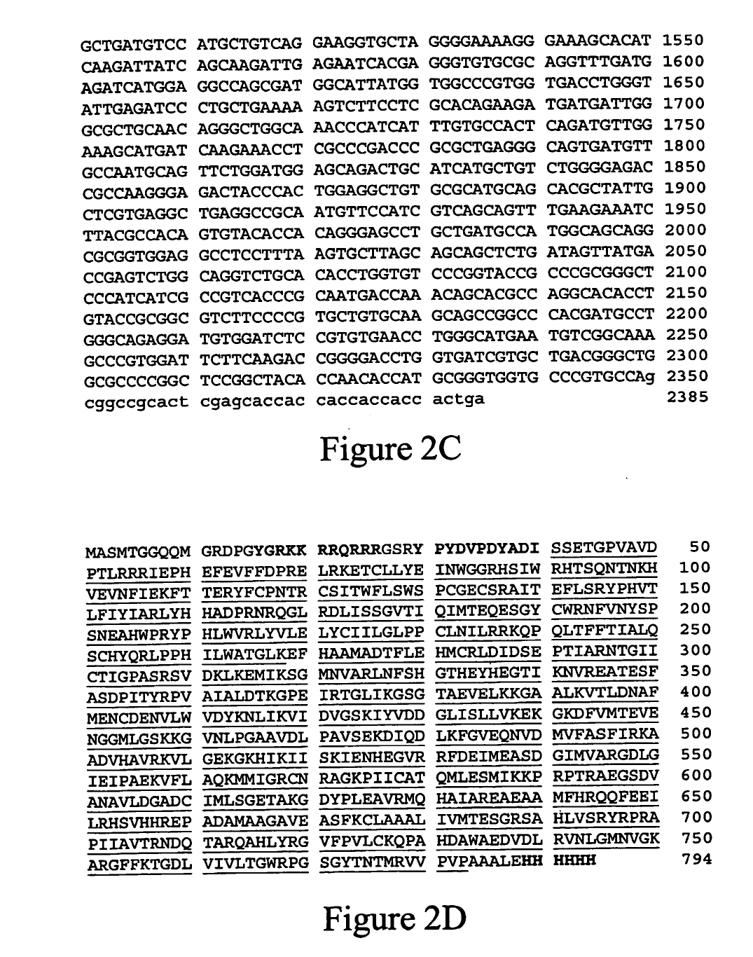

An exemplary first chimeric protein of the present invention which is suitable for use in rats, designated TAT-rAPOBEC-CMPK, is set forth in FIG. 2A. This first chimeric protein (rat) includes: an N-terminal HIV tat protein transduction domain, a hemagglutinin domain, a polypeptide fragment of rat APOBEC-1, a CMPK domain, and a C-terminal His tag. The amino acid sequence (SEQ ID No: 4) and encoding nucleotide sequence (SEQ ID No: 3) of this exemplary first chimeric protein (rat) is set forth in FIGS. 2D and 2B-C, respectively.

According to a second aspect of the present invention, a second chimeric protein is provided for use in combination with the first chimeric protein described above. The second chimeric protein includes a first polypeptide that includes a protein transduction domain and a second polypeptide the includes ACF or a fragment thereof which can bind to apolipoprotein B mRNA.

The first polypeptide of the second chimeric protein can be a protein transduction domain of the type described above. The protein transduction domain of the second chimeric protein can be the same or different from the protein transduction domain of the first chimeric protein.

The second polypeptide of the second chimeric protein, as noted above, includes ACF or a fragment thereof which can bind to apolipoprotein B mRNA. Although it has been proposed that a number of different proteins assist APOBEC-1 in editing apolipoprotein B mRNA, ACF has been identified as the minimal protein complement for editing in vitro in the human system (Mehta et al., Molecular cloning of apobec-1 complementation factor, a novel RNA binding protein involved in the editing of apo B mRNA,” Mol. Cell. Biol. 20:1846-1854 (2000), which is hereby incorporated by reference in its entirety). In accordance with the present invention, therefore, the second chimeric protein binds apolipoprotein B mRNA at the mooring sequence and through its interactions with the first chimeric protein, sequesters the first chimeric protein to the cytidine of the apolipoprotein B mRNA to be edited (i.e., at position 6666), thereby resulting in its conversion to a uridine. As noted above, this conversion results in a stop codon that contributes to expression of the apolipoprotein B48 derivative.

Recent studies have suggested that APOBEC-1 requires a chaperone for its nuclear localization (Yang et al., “Intracellular trafficking determinants in APOBEC-1, the catalytic subunit for cytidine to uridine editing of apolipoprotein B mRNA,” Exp. Cell Res. 267:153-164 (2001), which is hereby incorporated by reference in its entirety). More recently, however, it has been learned that APOBEC-1 is most likely associated with ACF throughout the cell and, therefore, it may import to the nucleus as an APOBEC-1/ACF complex. A bipartite nuclear localization signal is predicted in ACF (see below).

ACF is expressed at sufficient levels within the hepatic cells of rat (Dance et al., “Two proteins essential for apolipoprotein B mRNA editing are expressed from a single gene through alternative splicing,” J. Biol. Chem., electronically published as manuscript M111337200 (2002), which is hereby incorporated by reference in its entirety), such that augmenting of the intracellular ACF concentration is not needed. However, to optimize apolipoprotein B mRNA editing, in some instances it may be desirable to increase the intracellular concentration of ACF.

The full length rat ACF has an amino acid sequence according to SEQ ID No: 21 as follows:

```
Met Glu Ser Asn His Lys Ser Gly Asp Gly Leu Ser Gly Thr Gln Lys
    1 5 10 15
Glu Ala Ala Leu Arg Ala Leu Val Gln Arg Thr Gly Tyr Ser Leu Val
                          20 25 30
Gln Glu Asn Gly Gln Arg Lys Tyr Gly Gly Pro Pro Pro Gly Trp Asp
                  35 40 45
Thr Thr Pro Pro Glu Arg Gly Cys Glu Ile Phe Ile Gly Lys Leu Pro
          50 55 60
Arg Asp Leu Phe Glu Asp Glu Leu Ile Pro Leu Cys Glu Lys Ile Gly
  65 70 75 80
Lys Ile Tyr Glu Met Arg Met Met Met Asp Phe Asn Gly Asn Asn Arg
                                  85 90 95
Gly Tyr Ala Phe Val Thr Phe Ser Asn Lys Gln Glu Ala Lys Asn Ala
                        100 105 110
Ile Lys Gln Leu Asn Asn Tyr Glu Ile Arg Asn Gly Arg Leu Leu Gly
                115 120 125
Val Cys Ala Ser Val Asp Asn Cys Arg Leu Phe Val Gly Gly Ile Pro
        130 135 140
Lys Thr Lye Lys Arg Glu Glu Ile Leu Ser Glu Met Lys Lye Val Thr
145 150 155 160
Glu Gly Val Val Asp Val Ile Val Tyr Pro Ser Ala Ala Asp Lys Thr
                                165 170 175
Lys Asn Arg Gly Phe Ala Phe Val Glu Tyr Glu Ser His Arg Ala Ala
                        180 185 190
Ala Met Ala Arg Arg Arg Leu Leu Pro Gly Arg Ile Gln Leu Trp Gly
                195 200 205
His Pro Ile Ala Val Asp Trp Ala Glu Pro Glu Val Glu Val Asp Glu
        210 215 220
Asp Thr Met Ser Ser Val Lys Ile Leu Tyr Val Arg Asn Leu Met Leu
225 230 235 240
Ser Thr Ser Glu Glu Met Ile Glu Lys Glu Phe Asn Ser Ile Lys Pro
                                245 250 255
Gly Ala Val Glu Arg Val Lys Lys Ile Arg Asp Tyr Ala Phe Val His
                        260 265 270
Phe Ser Asn Arg Glu Asp Ala Val Glu Ala Met Lys Ala Leu Asn Gly
                275 280 285
Lys Val Leu Asp Gly Ser Pro Ile Glu Val Thr Leu Ala Lys Pro Val
        290 295 300
Asp Lys Asp Ser Tyr Val Arg Tyr Thr Arg Gly Thr Gly Gly Arg Asn
305 310 215 320
Thr Met Leu Gln Glu Tyr Thr Tyr Pro Leu Ser His Val Tyr Asp Pro
                                325 330 335
Thr Thr Thr Tyr Leu Gly Ala Pro Val Phe Tyr Thr Pro Gln Ala Tyr
                        340 345 350
Ala Ala Ile Pro Ser Leu His Phe Pro Ala Thr Lys Gly His Leu Ser
                355 360 365
Asn Arg Ala Leu Ile Arg Thr Pro Ser Val Arg Glu Ile Tyr Met Asn
        370 375 380
Val Pro Val Gly Ala Ala Gly Val Arg Gly Leu Gly Gly Arg Gly Tyr
385 390 395 400
Leu Ala Tyr Thr Gly Leu Gly Arg Gly Tyr Gln Val Lys Gly Asp Lys
                                405 410 415
Arg Gln Asp Lys Leu Tyr Asp Leu Leu Pro Gly Met Glu Leu Thr Pro
                        420 425 430
Met Asn Thr Ile Ser Leu Lys Pro Gln Gly Val Lys Leu Ala Pro Gln
                435 440 445
Ile Leu Glu Glu Ile Cys Gln Lys Asn Asn Trp Gly Gln Pro Val Tyr
        450 455 460
Gln Leu His Ser Ala Ile Gly Gln Asp Gln Arg Gln Leu Phe Leu Tyr
465 470 475 480
Lys Val Thr Ile Pro Ala Leu Ala Ser Gln Asn Pro Ala Ile His Pro
                                485 490 495
Phe Thr Pro Pro Lys Leu Ser Ala Tyr Val Asp Glu Ala Lys Arg Tyr
                        500 505 510
Ala Ala Glu His Thr Leu Gln Thr Leu Gly Ile Pro Thr Glu Gly Gly
                515 520 525
Asp Ala Gly Thr Thr Ala Pro Thr Ala Thr Ser Ala Thr Val Phe Pro
        530 535 540
Gly Tyr Ala Val Pro Ser Ala Thr Ala Pro Val Ser Thr Ala Gln Leu
545 550 555 560
Lys Gln Ala Val Thr Leu Gly Gln Asp Leu Ala Ala Tyr Thr Thr Tyr
                                565 570 575
Glu Val Tyr Pro Thr Phe Ala Val Thr Thr Arg Gly Asp Gly Tyr Gly
                        580 585 590
Thr Phe
```

A DNA molecule encoding the full length rat ACF has a nucleotide sequence according to SEQ ID No: 22 as follows:

| | |
|---|---|
| atggaatcaa atcacaaatc cggggatgga ttgagcggca cccagaagga agcagcactc | 60 |
| cgcgcactgg tccagcgcac aggatatagc ttggtccagg aaaatggaca aagaaaatat | 120 |
| ggtggtcctc caccaggctg ggatactaca cccccagaaa ggggctgcga gattttcatt | 180 |
| gggaaacttc cccgggacct ttttgaggat gaactcatac cattgtgtga aaaaattggt | 240 |
| aaaatttatg aaaigagaat gatgatggat ttcaatggga acaacagagg ctatgcattt | 300 |
| gtaaccttct caaataagca ggaagccaag aatgcaatca agcaacttaa taattatgaa | 360 |
| attcggaatg gccgtctcct gggcgtctgt gccagtgtgg acaactgccg gttgtttgtg | 420 |
| gggggaatcc ccaaaaccaa aaagagagaa gaaatcttgt cagagatgaa aaaggtcact | 480 |
| gaaggagttg ttgatgtcat tgtctaccca agcgctgccg ataaaaccaa aaaccggggg | 540 |
| tttgcctttg tggaatatga gagtcaccgc gcagccgcca tggctaggcg gaggctgctg | 600 |
| ccaggaagaa ttcagttgtg gggacatcct atcgcagtag actgggcaga gccagaagtc | 660 |
| gaagttgacg aagacacaat gtcttccgtg aaaatcctgt acgtaaggaa ccttatgctg | 720 |
| tctacctcgg aagagatgat tgagaaggaa ttcaacagta ttaaaccagg tgctgtggaa | 780 |
| cgggtgaaga agatccgaga ctatgctttt gtgcatttca gtaaccgaga agatgcagtt | 840 |
| gaagccatga aggctttgaa tggcaaggtg ctggatggtt ccccaataga agtgaccttg | 900 |
| gccaagccag tggacaagga cagttacgtt aggtacaccc ggggcaccgg gggcaggaac | 960 |
| accatgctgc aagaatacac ctaccctctg agccatgttt atgaccctac cacaacctac | 1020 |
| cttggagctc ctgtcttcta tactccccaa gcctacgcag ccattccaag tcttcatttc | 1080 |
| ccagctacca aaggacatct cagcaacaga gctctcatcc ggaccccttc tgtcagagaa | 1140 |
| atttacatga atgtccctgt aggggctgcg ggcgtgagag gactgggcgg ccgtgggtat | 1200 |
| ttggcatata caggcctggg tcgaggatac caggtcaaag gagacaagag acaagacaaa | 1260 |
| ctctatgacc ttctgcctgg gatggagctc accccgatga atactatctc tttaaaacca | 1320 |
| caaggagtta aacttgctcc tcagatatta gaagaaatct gtcagaaaaa taactgggga | 1380 |
| cagccagtgt accagctgca ctctgccatt ggacaagacc aaagacagtt attcctatac | 1440 |
| aaagtaacta tcccagcgct ggccagccag aatcctgcga tccacccttt cacaccccca | 1500 |
| aagctaagcg cctacgtgga tgaagcaaag aggtacgccg cagagcacac cctacagaca | 1560 |
| ctaggcatcc ccacagaagg aggggacgct gggactacag cacccactgc cacatccgcc | 1620 |
| actgtgtttc caggatacgc tgtccccagt gccaccgctc ctgtgtctac agcccagctc | 1680 |
| aagcaagcag tgacacttgg acaagactta gcagcatata caacctatga ggtctaccct | 1740 |
| acttttgcag tgaccacccg aggtgatgga tatggcacct tctga 1785 | |

The amino acid sequence and nucleotide sequence for the full length rat ACF65 is reported at Genbank Accession Nos. AAK50145 and AY028945, respectively, each of which is hereby incorporated by reference in its entirety. In addition, it should be noted that a short isoform of rat ACF64 exists, as identified at Genbank Accession No. AF290984, which is hereby incorporated by reference in its entirety.

The full length human ACF has an amino acid sequence according to SEQ ID No: 23 as follows:

```
Met Glu Ser Asn His Lys Ser Gly Asp Gly Leu Ser Gly Thr Gln Lys
    1 5 10 15
Glu Ala Ala Leu Arg Ala Leu Val Gln Arg Thr Gly Tyr Ser Leu Val
                          20 25 30
Gln Glu Asn Gly Gln Arg Lys Tyr Gly Gly Pro Pro Pro Gly Trp Asp
                  35 40 45
Ala Ala Pro Pro Glu Arg Gly Cys Glu Ile Phe Ile Gly Lys Leu Pro
          50 55 60
Arg Asp Leu Phe Glu Asp Glu Leu Ile Pro Leu Cys Glu Lys Ile Gly
  65 70 75 80
Lys Ile Tyr Glu Met Arg Met Met Met Asp Phe Asn Gly Asn Asn Arg
                                  85 90 95
Gly Tyr Ala Phe Val Thr Phe Ser Asn Lys Val Glu Ala Lys Asn Ala
                        100 105 110
Ile Lys Gln Leu Asn Asn Tyr Glu Ile Arg Asn Gly Arg Leu Leu Gly
                115 120 125
Val Cys Ala Ser Val Asp Asn Cys Arg Leu Phe Val Gly Gly Ile Pro
        130 135 140
Lys Thr Lys Lys Arg Glu Glu Ile Leu Ser Glu Met Lys Lys Val Thr
145 150 155 160
Glu Gly Val Val Asp Val Ile Val Tyr Pro Ser Ala Ala Asp Lys Thr
                                165 170 175
Lys Asn Arg Gly Phe Ala Phe Val Glu Tyr Glu Ser His Arg Ala Ala
                        180 185 190
Ala Met Ala Arg Arg Lys Leu Leu Pro Gly Arg Ile Gln Leu Trp Gly
                195 200 205
His Gly Ile Ala Val Asp Trp Ala Glu Pro Glu Val Glu Val Asp Glu
        210 215 220
Asp Thr Met Ser Ser Val Lys Ile Leu Tyr Val Arg Asn Leu Met Leu
225 230 235 240
Ser Thr Ser Glu Glu Met Ile Glu Lys Glu Phe Asn Asn Ile Lys Pro
                                245 250 255
Gly Ala Val Glu Arg Val Lys Lys Ile Arg Asp Tyr Ala Phe Val His
                        260 265 270
Phe Ser Asn Arg Lys Asp Ala Val Glu Ala Met Lys Ala Leu Asn Gly
                275 280 285
Lys Val Leu Asp Gly Ser Pro Ile Glu Val Thr Leu Ala Lys Pro Val
        290 295 300
Asp Lys Asp Ser Tyr Val Arg Tyr Thr Arg Gly Thr Gly Gly Arg Gly
305 310 315 320
Thr Met Leu Gln Gly Glu Tyr Thr Tyr Ser Leu Gly Gln Val Tyr Asp
                                325 330 335
Pro Thr Thr Thr Tyr Leu Gly Ala Pro Val Phe Tyr Ala Pro Gln Thr
                        340 345 350
Tyr Ala Ala Ile Pro Ser Leu His Phe Pro Ala Thr Lys Gly His Leu
                335 360 365
Ser Asn Arg Ala Ile Ile Arg Ala Pro Ser Val Arg Gly Ala Ala Gly
        370 375 380
Val Arg Gly Leu Gly Gly Arg Gly Tyr Leu Ala Tyr Thr Gly Leu Gly
385 390 395 400
Arg Gly Tyr Gln Val Lys Gly Asp Lys Arg Glu Asp Lys Leu Tyr Asp
                                405 410 415
Ile Leu Pro Gly Met Glu Leu Thr Pro Met Asn Pro Val Thr Leu Lys
                        420 425 430
Pro Gln Gly Ile Lys Leu Ala Pro Gln Ile Leu Glu Glu Ile Cys Gln
                435 440 445
Lys Asn Asn Trp Gly Gln Pro Val Tyr Gln Leu His Ser Ala Ile Gly
        450 455 460
Gln Asp Gln Arg Gln Leu Phe Leu Tyr Lys Ile Thr Ile Pro Ala Leu
465 470 475 480
Ala Ser Gln Asn Pro Ala Ile His Pro Phe Thr Pro Pro Lys Leu Ser
                                485 490 495
Ala Phe Val Asp Glu Ala Lys Thr Tyr Ala Ala Glu Tyr Thr Leu Gln
                        500 505 510
Thr Leu Gly Ile Pro Thr Asp Gly Gly Asp Gly Thr Met Ala Thr Ala
                515 520 525
Ala Ala Ala Ala Thr Ala Phe Pro Gly Tyr Ala Val Pro Asn Ala Thr
        530 535 540
Ala Pro Val Ser Ala Ala Gln Leu Lys Gln Ala Val Thr Leu Gly Gln
545 550 555 560
Asp Leu Ala Ala Tyr Thr Thr Tyr Glu Val Tyr Pro Thr Phe Ala Val
                                565 570 575
Thr Ala Arg Gly Asp Gly Tyr Gly Thr Phe
                        580 585
```

A DNA molecule encoding the full length human ACF has a nucleotide sequence according to SEQ ID No: 24 as follows:

| | |
|---|---|
| atggaatcaa atcacaaatc cggggatgga ttgagcggca ctcagaagga agcagccctc | 60 |
| cgcgcactgg tccagcgcac aggatatagc ttggtccagg aaaatggaca aagaaaatat | 120 |
| ggtggccctc cacctggttg ggatgctgca ccccctgaaa ggggctgtga aatttttatt | 180 |
| ggaaaacttc cccgagacct ttttgaggat gagcttatac cattatgtga aaaaatcggt | 240 |
| aaaatttatg aaatgagaat gatgatggat tttaatggca acaatagagg atatgcattt | 300 |
| gtaacatttt caaataaagt ggaagccaag aatgcaatca agcaacttaa taattatgaa | 360 |
| attagaaatg ggcgcctctt aggggtttgt gccagtgtgg acaactgccg attatttgtt | 420 |
| gggggcatcc caaaaaccaa aaagagagaa gaaatcttat cggagatgaa aaaggttact | 480 |
| gaaggtgttg tcgatgtcat cgtctaccca agcgctgcag ataaaaccaa aaaccgaggc | 540 |
| tttgccttcg tggagtatga gagtcatcga gcagctgcca tggcgaggag gaaactgcta | 600 |
| ccaggaagaa ttcagttatg gggacatggt attgcagtag actgggcaga gccagaagta | 660 |
| gaagtigatg aagatacaat gtcttcagtg aaaatcctat atgtaagaaa tcttatgctg | 720 |
| tctacctctg aagagatgat tgaaaaggaa ttcaacaata tcaaaccagg tgctgtggag | 780 |
| agggtgaaga aaattcgaga ctatgctttt gtgcacttca gtaaccgaaa agatgcagtt | 840 |
| gaggctatga aagctttaaa tggcaaggtg ctggatggtt cccccattga agtcacccta | 900 |
| gcaaaaccag tggacaagga cagttatgtt aggtataccc gaggcacagg tggaaggggc | 960 |
| accatgctgc aaggagagta tacctactct ttgggccaag tttatgatcc caccacaacc | 1020 |
| taccttggag ctcctgtctt ctatgccccc cagacctatg cagcaattcc cagtcttcat | 1080 |
| ttcccagcca ccaaaggaca tctcagcaac agagccatta tccgagcccc ttctgttaga | 1140 |
| ggggctgcgg gagtgagagg actgggcggc cgtggctatt tggcatacac aggcctgggt | 1200 |
| cgaggatacc aggtcaaagg agacaaaaga gaagacaaac tctatgacat tttacctggg | 1260 |
| atggagctca ccccaatgaa tcctgtcaca ttaaaacccc aaggaattaa actcgctccc | 1320 |
| cagatattag aagagatttg tcagaaaaat aactggggac agccagtgta ccagctgcac | 1380 |
| tctgctattg gacaagacca aagacagcta ttcttgtaca aaataactat tcctgctcta | 1440 |
| gccagccaga atcctgcaat ccaccctttc acacctccaa agctgagtgc ctttgtggat | 1500 |
| gaagcaaaga cgtatgcagc cgaatacacc ctgcagaccc tgggcatccc cactgatgga | 1560 |
| ggcgatggca ccatggctac tgctgctgct gctgctactg ctttcccagg atatgctgtc | 1620 |
| cctaatgcaa ctgcacccgt gtctgcagcc cagctcaagc aagcggtaac ccttggacaa | 1680 |
| gacttagcag catatacaac ctatgaggtc tacccaactt ttgcagtgac tgcccgaggg | 1740 |
| gatggatatg gcaccttctg a 1761 | |

The amino acid sequence and nucleotide sequence for the full length human ACF is reported at Genbank Accession Nos. AAF76221 and AF271789, respectively, each of which is hereby incorporated by reference in its entirety.

In comparing the human and rat ACF homologs, it is apparent that these proteins share 93.5 percent identity at the amino acid level and, moreover, antibodies raised against the human ACF also recognize rat ACF. Is has been reported that functional complementation of apolipoprotein B mRNA editing by APOBEC-1 involves the N-terminal 380 residues of ACF (Blanc et al., “Mutagenesis of Apobec-1 complementation factor reveals distinct domains that modulate RNA binding, protein-protein interaction with Apobec-1, and complementation of C to U RNA-editing activity,” J. Biol. Chem 276(49): 46386-46393 (2001), which is hereby incorporated by reference in its entirety).

The second chimeric protein of the present invention can also include one or more other polypeptide sequences, including without limitation: (i) a polypeptide that includes a cytoplasmic localization protein or a fragment thereof which, upon cellular uptake of the second chimeric protein, localizes the second chimeric protein to the cytoplasm; (ii) a polypeptide that includes a plurality of adjacent histidine residues; and (iii) a polypeptide that includes a hemagglutinin domain. Each of these has been described above with respect to the first chimeric protein.

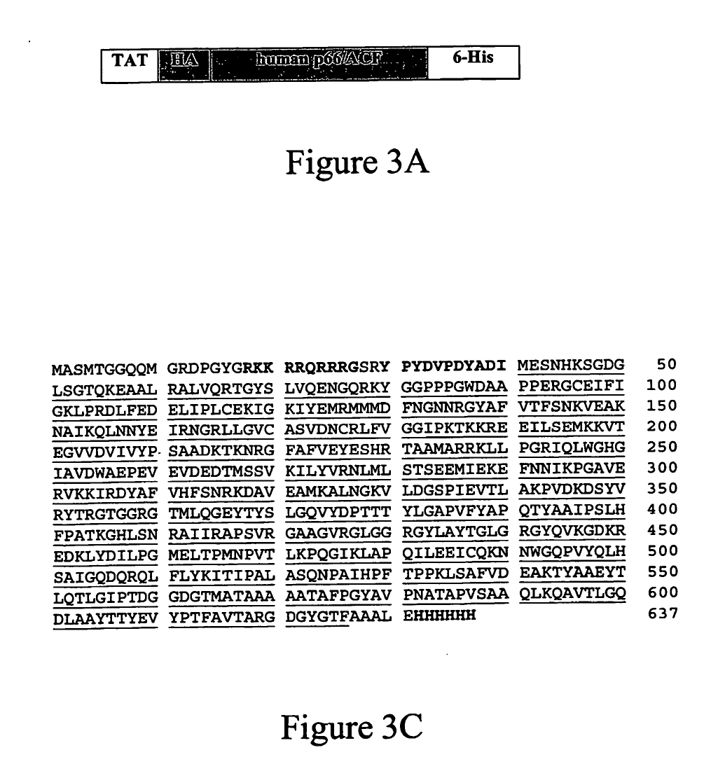

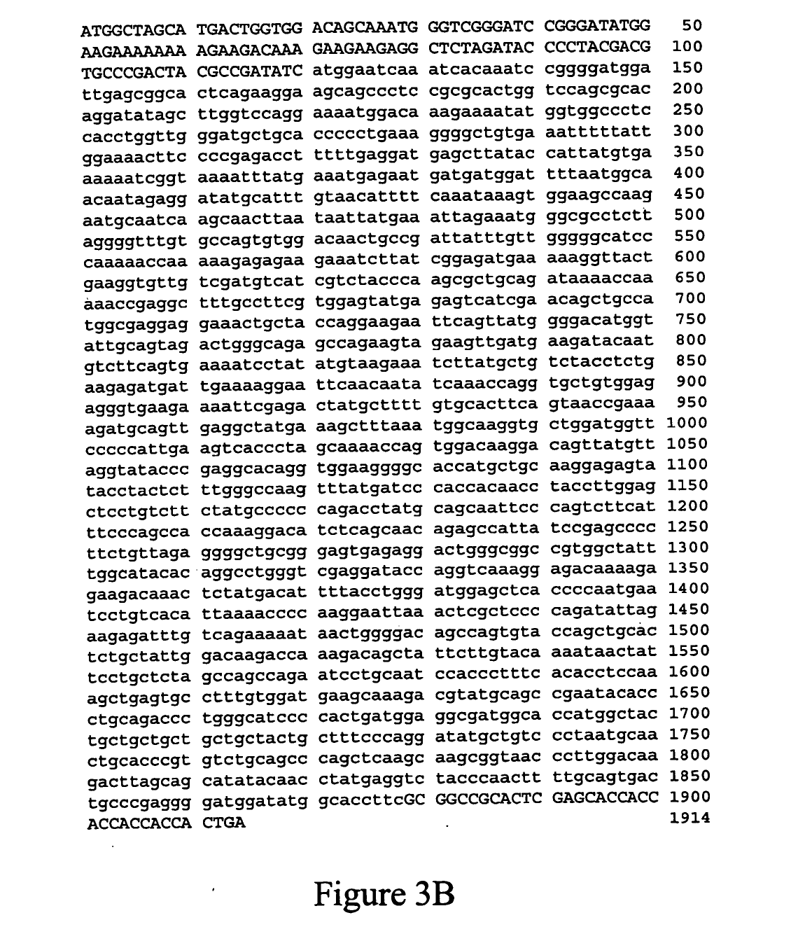

An exemplary second chimeric protein of the present invention which is suitable for use in humans, designated TAT-hACF, is set forth in FIG. 3A. This second chimeric protein (human) includes: an N-terminal HIV tat protein transduction domain, a hemagglutinin domain, a polypeptide fragment of human. ACF, and a C-terminal His tag. The amino acid sequence (SEQ ID No: 6) and encoding nucleotide sequence (SEQ ID No: 5) of this exemplary second chimeric protein (human) is set forth in FIGS. 3B-C.

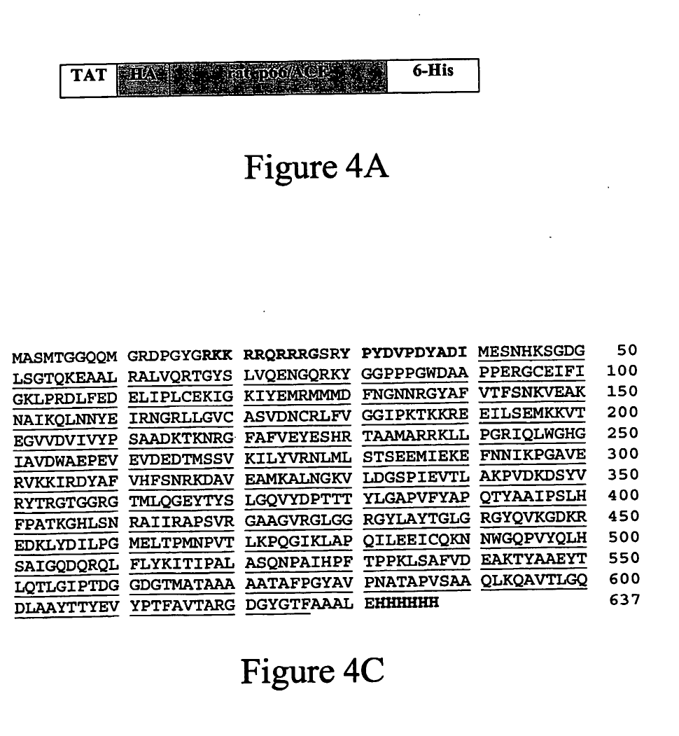

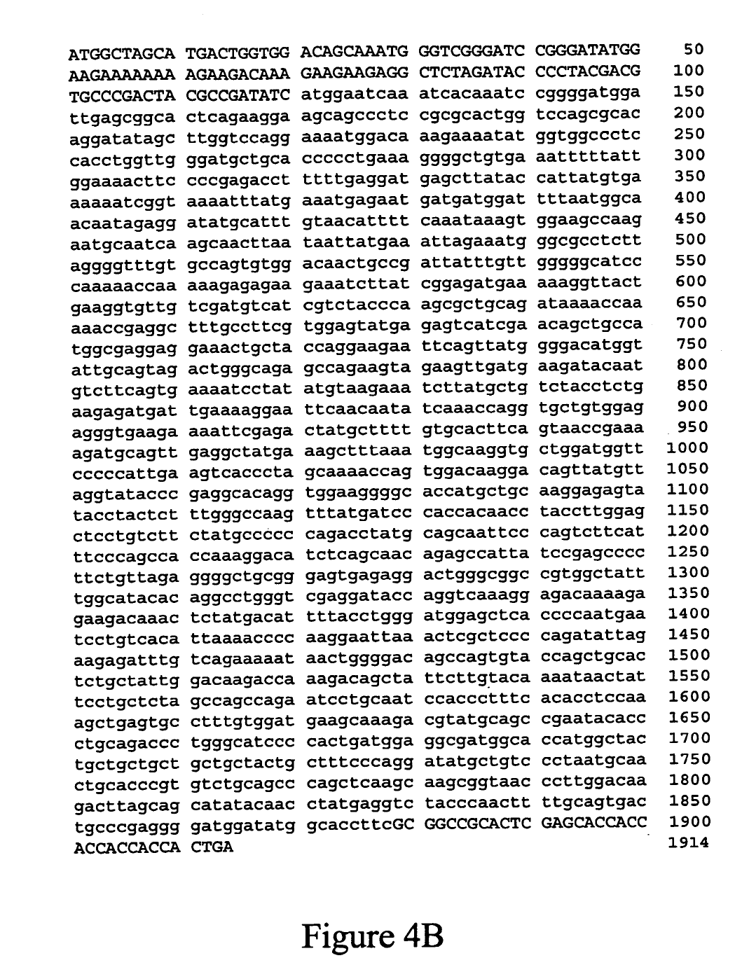

An exemplary second chimeric protein of the present invention which is suitable for use in rats, designated TAT-rACF, is set forth in FIG. 4A. This second chimeric protein (rat) includes: an N-terminal HIV tat protein transduction domain, a hemagglutinin domain, a polypeptide fragment of rat ACF, and a C-terminal His tag. The amino acid sequence (SEQ ID No: 8) and encoding nucleotide sequence (SEQ ID No: 7) of this exemplary second chimeric protein (rat) is set forth in FIGS. 4B-C.

DNA molecules encoding the above-identified first and second chimeric proteins can be assembled using conventional molecular genetic manipulation for subcloning gene fragments, such as described by Sambrook et al., Molecular Cloning: A Laboratory Manual, Cold Springs Laboratory, Cold Springs Harbor, N.Y. (1989), and Ausubel et al. (ed.), Current Protocols in Molecular Biology, John Wiley & Sons (New York, N.Y.) (1999 and preceding editions), each of which is hereby incorporated by reference in its entirety. In conjunction therewith, desired fragments of the APOBEC-1, ACF, or CMPK encoding DNA molecules can be obtained using the PCR technique together with specific sets of primers chosen to represent particular portions of the protein. Erlich et al., Science 252:1643-51 (1991), which is hereby incorporated by reference in its entirety.

Once the desired DNA molecules have been assembled, DNA constructs can be assembled by ligating together the DNA molecule encoding the first or second chimeric protein with appropriate regulatory sequences including, without limitation, a promoter sequence operably connected 5′ to the DNA molecule, a 3′ regulatory sequence operably connected 3′ of the DNA molecule, as well as any enhancer elements, suppressor elements, etc. The DNA construct can then be inserted into an appropriate expression vector. Thereafter, the vector can be used to transform a host cell, typically although not exclusively a prokaryote, and the recombinant host cell can express the first or second chimeric protein of the present invention.

When a prokaryotic host cell is selected for subsequent transformation, the promoter region used to construct the DNA construct (i.e., transgene) should be appropriate for the particular host. The DNA sequences of eukaryotic promoters, as described infra for expression in eukaryotic host cells, differ from those of prokaryotic promoters. Eukaryotic promoters and accompanying genetic signals may not be recognized in or may not function in a prokaryotic system and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology, 68:473 (1979), which is hereby incorporated by reference in its entirety.

Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires a Shine-Dalgarno (“SD”) sequence about 7-9 bases 5′ to the initiation codon (“ATG”) to provide a ribosome binding site. Thus, any SD-ATG combination that can be utilized by host cell ribosomes may be employed. Such combinations include, but are not limited to, the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

Mammalian cells can also be used to recombinantly produce the first or second chimeric proteins of the present invention. Suitable mammalian host cells include, without limitation: COS (e.g., ATCC No. CRL 1650 or 1651), BHK (e.g., ATCC No. CRL 6281), CHO (ATCC No. CCL 61), HeLa (e.g., ATCC No. CCL 2), 293 (ATCC No. 1573), CHOP, and NS-1 cells. Suitable expression vectors for directing expression in mammalian cells generally include a promoter, as well as other transcription and translation control sequences known in the art. Common promoters include, without limitation, SV40, MMTV, metallothionein-1, adenovirus Ela, CMV, immediate early, immunoglobulin heavy chain promoter and enhancer, and RSV-LTR.

Regardless of the selection of host cell, once the DNA molecule coding for a first or second chimeric protein has been ligated to its appropriate regulatory regions using well known molecular cloning techniques, it can then be introduced into a suitable vector or otherwise introduced directly into a host cell using transformation protocols well known in the art (Sambrook et al., Molecular Cloning: A Laboratory Manual, Second Edition, Cold Spring Harbor Press, NY (1989), which is hereby incorporated by reference in its entirety).

The recombinant DNA molecule can be introduced into host cells via transformation, particularly transduction, conjugation, mobilization, or electroporation. Suitable host cells include, but are not limited to, bacteria, virus, yeast, mammalian cells, insect, plant, and the like. The host cells, when grown in an appropriate medium, are capable of expressing the chimeric protein, which can then be isolated therefrom and, if necessary, purified. The first or second chimeric protein is preferably produced in purified form (preferably at least about 80%, more preferably 90%, pure) by conventional techniques, including immuno-purification techniques. Immuno-isolation followed by metal-chelating affinity chromatography and cationic exchange chromatography is described in Example 1 infra.

A further aspect of the present invention relates to a number of compositions, preferably pharmaceutical compositions, which include the first and/or second chimeric protein of the present invention.

According to one embodiment, a composition includes a pharmaceutically acceptable carrier and the first chimeric protein of the present invention. The first chimeric protein is preferably present in an amount which is effective to modify apolipoprotein B mRNA editing in cells which uptake the first chimeric protein.

According to a second embodiment, a composition includes the first and second chimeric proteins of the present invention. This composition can also include a pharmaceutically acceptable carrier in which the first and second chimeric proteins are dispersed. Preferably, the first chimeric protein is present in an amount which is effective to modify apolipoprotein B mRNA editing in cells which uptake the first chimeric protein and the second chimeric protein is present in an amount which is effective to bind apolipoprotein B mRNA and assist the first chimeric protein in modifying apolipoprotein B mRNA in cells which uptake the first and second chimeric proteins.

The compositions of the present invention can also include suitable excipients, or stabilizers, and can be in solid or liquid form such as, tablets, capsules, powders, solutions, suspensions, or emulsions. Typically, the compositions will contain from about 0.01 to 99 percent, preferably from about 20 to 75 percent of the chimeric protein(s), together with the carrier, excipient, stabilizer, etc.

The solid unit dosage forms can be of the conventional type. The solid form can be a capsule, such as an ordinary gelatin type containing the first and/or second chimeric protein(s) of the present invention and a carrier, for example, lubricants and inert fillers such as, lactose, sucrose, or cornstarch. In another embodiment, these first and/or second chimeric protein(s) are tableted with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The first and/or second chimeric protein(s) of the present invention may also be administered in injectable or topically-applied dosages by solution or suspension of these materials in a physiologically acceptable diluent with a pharmaceutical carrier. Such carriers include sterile liquids, such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

For use as aerosols, the first and/or second chimeric protein(s) of the present invention in solution or suspension may be packaged in a pressurized aerosol container together with suitable propellants, for example, hydrocarbon propellants like propane, butane, or isobutane with conventional adjuvants. The compositions of the present invention also may be administered in a non-pressurized form such as in a nebulizer or atomizer.

Depending upon the treatment being effected, the compounds of the present invention can be administered orally, topically, transdermally, parenterally, subcutaneously, intravenously, intramuscularly, intraperitoneally, by intracavitary or intravesical instillation, intraocularly, intraarterially, intralesionally, or by application to mucous membranes, such as, that of the nose, throat, and bronchial tubes. In most instances, subcutaneous, intravenous, intramuscular, intraperitoneal, and intraarterial routes are preferred.

Compositions within the scope of this invention include all compositions wherein the first and/or second chimeric proteins of the present invention is contained in an amount effective to achieve its intended purpose, noted above. While individual needs vary, determination of optimal ranges of effective amounts of each of the first and second chimeric proteins is within the skill of the art. Typical dosages comprise about 0.01 to about 100 mg/kg·body wt. The preferred dosages comprise about 0.1 to about 100 mg/kg·body wt. The most preferred dosages comprise about 1 to about 100 mg/kg·body wt.

The amounts of the first and second chimeric proteins can be determined by one of ordinary skill in the art using routine testing to optimize the dosage levels of the first and second chimeric proteins in accordance with the desired degree of apolipoprotein B mRNA editing. Based on May 2001 guidelines by the National Institutes of Health's National Cholesterol Education Program (NCEP), individuals at low risk for a heart attack should have LDL levels under 160 mg/dL, while those at highest risk should aim for LDLs under 100 mg/dL. Treatment regimen for the administration of the first and/or second chimeric proteins of the present invention can also be determined readily by those with ordinary skill in art.

Typically, the first and/or second chimeric proteins (or compositions which contain one or both of the chimeric proteins of the present invention) can be administered via a drug delivery device which includes a chimeric protein or a composition of the present invention. Exemplary delivery devices include, without limitation, liposomes, niosomes, transdermal patches, implants, and syringes.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989), which is hereby incorporated by reference in its entirety). When liposomes are endocytosed by a target cell, for example, they can be routed to acidic endosomes which will destabilize the liposome and result in drug release.

Alternatively, the liposome membrane can be chemically modified such that an enzyme is placed as a coating on the membrane which slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release.

This liposome delivery system can also be made to accumulate at a target organ, tissue, or cell via active targeting. In accordance with the present invention, liposomes can be targeted to liver cells by incorporating into the liposome bilayer a molecule which target hepatocyte receptors. One such molecule is the asialoglycoprotein asialofetuin, which targets the asialoglycoprotein receptor of hepatocytes. The incorporation of asialofetuin into the liposome bilayer can be performed according to the procedures set forth in Wu et al., “Increased liver uptake of liposomes and improved targeting efficacy by labeling with asialofetuin in rodents,” Hepatology 27(3):772-778 (1998), which is hereby incorporated by reference in its entirety.

Niosomes are vesicles formed by amphiphilic materials. Non-ionic surfactants were the first materials studied (Iga et al., “Membrane modification by negatively charged stearylpolyoxyethylene derivatives for thermosensitive liposomes: Reduced liposomal aggregation and avoidance of reticuloendothelial system uptake,” J. Drug Target 2:259-67 (1994), which is hereby incorporated by reference in its entirety) and a large number of surfactants have since been found to self assemble into closed bilayer vesicles (Ahl et al., “Enhancement of the in vivo circulation lifetime of L-alpha-distearoylphosphatidylcholine liposomes: Importance of liposomal aggregation versus complement opsonization,” Biochem Biophys Acta 1329:370-82 (1997), which is hereby incorporated by reference in its entirety). These niosomal materials may be used for delivery of the first or second chimeric protein or for delivery of APOBEC-1 or fragments thereof alone or in combination with ACF or fragments thereof

For example, 200 nm doxorubicin niosomes with a polyoxyethylene (molecular weight 1,000) surface have been shown to be rapidly taken up by the liver (Uchegbu et al., “Distribution, metabolism and tumoricidal activity of doxorubicin administered in sorbitan monostearate (Span 60) niosomes in the mouse,” Pharm. Res. 12:1019-24 (1995), which is hereby incorporated by reference in its entirety), allowing polymeric drug conjugates to be formed for delivery of the drug (see Duncan, “Drug polymer conjugates—potential for improved chemotherapy,” Anti-Cancer Drugs 3:175-210 (1992), which is hereby incorporated by reference in its entirety). These techniques can be readily adapted for delivery of the first and second chimeric proteins or, alternatively, APOBEC-1 or a fragment thereof alone or in combination with ACF or a fragment thereof

Compositions including the liposomes or niosomes in a pharmaceutically acceptable carrier are also contemplated.

Transdermal delivery devices have been employed for delivery of low molecular weight proteins by using lipid-based compositions (i.e., in the form of a patch) in combination with sonophoresis. However, as reported in U.S. Pat. No. 6,041,253 to Ellinwood, Jr. et al., which is hereby incorporated by reference in its entirety, transdermal delivery can be further enhanced by the application of an electric field, for example, by iontophoresis or electroporation. Using low frequency ultrasound which induces cavitation of the lipid layers of the stratum corneum, higher transdermal fluxes, rapid control of transdermal fluxes, and drug delivery at lower ultrasound intensities can be achieved. Still further enhancement can be obtained using a combination of chemical enhancers and/or magnetic field along with the electric field and ultrasound.

Implantable or injectable protein depot compositions can also be employed, providing long-term delivery of, e.g., the first and second chimeric proteins. For example, U.S. Pat. No. 6,331,311 to Brodbeck et al., which is hereby incorporated by reference in its entirety, reports an injectable depot gel composition which includes a biocompatible polymer, a solvent that dissolves the polymer and forms a viscous gel, and an emulsifying agent in the form of a dispersed droplet phase in the viscous gel. Upon injection, such a gel composition can provide a relatively continuous rate of dispersion of the agent to be delivered, thereby avoiding an initial burst of the agent to be delivered.

Other suitable protein delivery system which are known to those of skill in the art can also be employed to achieve the desired delivery and, thus, modification in the editing of apolipoprotein B mRNA and its concomitant effects.

By virtue of the first chimeric protein being able to edit apolipoprotein B mRNA, the present invention affords a method of modifying apolipoprotein B mRNA editing in vivo. This aspect of the present invention can be carried out by contacting apolipoprotein B mRNA in a cell with the first chimeric protein of the present invention under conditions effective to increase the concentration of apolipoprotein B48 which is secreted by the cell as compared to the concentration of apolipoprotein B 100 which is secreted by the cell, relative to an untreated cell (i.e., which has not taken up the first chimeric protein). Basically, the contacting is carried out by exposing the cell to the first chimeric protein under conditions effective to induce cellular uptake of the first chimeric protein. Because the first chimeric protein includes the first polypeptide (i.e., which includes a protein transduction domain), the first chimeric protein is taken up by the cell. In addition, the same cell can also be contacted with the second chimeric protein of the present invention, causing the second chimeric protein also to be taken up by the cell. As a result, the apolipoprotein B mRNA in the cell is contacted by the second chimeric protein, binding the apolipoprotein mRNA (as described above) so as to facilitate editing thereof by the first chimeric protein. The cell in which the apolipoprotein B mRNA editing is modified can be any cell which can synthesize and secrete VLDL with apolipoprotein B or its derivatives. Exemplary cells of this type include liver cells and intestinal cells, although preferably liver cells. The cell can also be in a mammal, preferably a human.

Likewise, the present invention also affords a method of reducing serum LDL levels. This aspect of the present invention can be carried out by delivering into one or more cells of a patient, without genetically modifying the cells, an amount of a protein comprising APOBEC-1 or a fragment thereof which can edit mRNA encoding apolipoprotein B, which amount is effective to increase the concentration of VLDL-apolipoprotein B48 that is secreted by the one or more cells into serum and, consequently, reduce the serum concentration of LDL. In accordance with this aspect of the present invention, the patient is a mammal, preferably a human, and the one or more cells are preferably liver cells, intestinal cells, or a combination thereof

To sustain the reduced serum LDL levels, delivery of the protein into the one or more cells is preferably repeated periodically (i.e., following a delay of from about 1 to about 7 days).

Delivery of the protein into the one or more cells can be carried out by exposing the one or more cells to the protein under conditions effective to cause cellular uptake of the protein. Preferably, the protein which includes APOBEC-1 or a fragment thereof is actually the first chimeric protein of the present invention and the protein transduction domain induces cellular uptake by the one or more cells. In addition to delivering the protein, a second protein can also be delivered simultaneously into the one or more cells of the patient, without genetically modifying the cells, where the second protein includes ACF or a fragment thereof which can bind to apolipoprotein B mRNA. Preferably, the second protein is the second chimeric protein of the present invention and the protein transduction domain induces cellular uptake by the one or more cells.

Alternatively, APOBEC-1 can be delivered directly into one or more liver cells by contacting each of them with liposomes including a molecule which binds to a hepatocyte receptor (e.g., asialofetuin), thereby inducing uptake of the liposomes and degradation thereof intracellularly to empty their contents into the one or more liver cells. In addition, ACF or a fragment thereof which can bind to apolipoprotein B mRNA can also be delivered via the liposomes.

By increasing the ratio of apolipoprotein B48 to apolipoprotein B 100 which is secreted by the one or more cells, the present invention also relates to a method of treating or preventing an atherogenic disease or disorder. This aspect of the present invention can be carried out by administering to a patient an effective amount of a protein comprising APOBEC-1 or a fragment thereof which can edit mRNA encoding apolipoprotein B, wherein upon said administering the protein is taken up by one or more cells of the patient that can synthesize and secrete VLDL-apolipoprotein under conditions which are effective to increase the concentration of VLDL-apolipoprotein B48 that is secreted by the one or more cells into serum, whereby rapid clearing of VLDL-apolipoprotein B48 from serum decreases the serum concentration of LDL to treat or prevent the atherogenic disease or disorder. In accordance with this aspect of the present invention, the patient is a mammal, preferably a human, and the one or more cells are preferably liver cells.

Administration of the protein can be carried out according to any of the above-identified approaches. Continued preventative or therapeutic treatment can be effected by repeatedly administering the APOBEC-1 protein periodically (i.e., following a delay of from about 1 to about 7 days).

Preferably, the protein which includes APOBEC-1 or a fragment thereof is actually the first chimeric protein of the present invention and the protein transduction domain induces cellular uptake by the one or more cells. As with the above-described methods, a second protein that includes ACF or a fragment thereof which can bind to apolipoprotein B mRNA can also be delivered simultaneously. Preferably, the second protein is the second chimeric protein of the present invention and the protein transduction domain induces cellular uptake by the one or more cells.

Alternatively, using a liposome delivery vehicle, APOBEC-1 and optionally ACF can be delivered directly into one or more liver cells by contacting each of them with a liposome including a molecule which binds to a hepatocyte receptor, thereby inducing uptake of the liposomes and degradation thereof intracellularly to empty their contents into the one or more liver cells.

### EXAMPLES

The following examples are intended to illustrate, but by no means are intended to limit, the scope of the present invention as set forth in the appended claims.

### Example 1

### Generation of TAT Fusion Protein

The induction of hepatic apolipoprotein B mRNA editing was sought through TAT mediated APOBEC-1 protein transduction into liver cells. It has been shown that linking an 11-amino-acid protein transduction domain (PTD) of HIV-1 TAT protein to heterologous protein conferred the ability to transduce into cells (Nagahara et al., “Transduction of full-length TAT fusion proteins into mammalian cells: TAT-p27^{Kip1} induces cell migration,” Nature Med. 4:1449-1452 (1998); Schwarze et al., “In vivo protein transduction: delivery of a biologically active protein into the mouse,” Science 285:1569-1572 (1999); Vocero-Akbani et al., “Killing HIV-infected cells by transduction with an HIV protease-activated caspase-3 protein,” Nature Med. 5:29-33 (1999), each of which is hereby incorporated by reference in its entirety). PTD-linked protein transduced into ˜100% of cells and the transduction process occurred in a rapid and concentration-dependent but receptor- and transporter-independent manner (Schwarze et al., “Protein transduction: unrestricted delivery into all cells,” Trends Cell Biol. 10:290-295 (2000), which is hereby incorporated by reference in its entirety). Liver cells have been shown to be susceptible to transduction (Nagahara et al., “Transduction of full-length TAT fusion proteins into mammalian cells: TAT-p27^{Kip1} induces cell migration,” Nature Med. 4:1449-1452 (1998), which is hereby incorporated by reference in its entirety). In order to produce in-frame TAT fusion protein from E. coli, a prokaryotic expression vector was constructed that has an N-terminal PTD flanked by glycine residues for free bond rotation of the domain (Schwarze et al., “In vivo protein transduction: delivery of a biologically active protein into the mouse,” Science 285:1569-1572 (1999), which is hereby incorporated by reference in its entirety), an hemagglutinin (HA) tag and a C-terminal 6-histidine tag. Using this vector as a backbone, a plasmid was constructed to encode full-length TAT-rAPOBEC-CMPK protein, SEQ ID No: 4 (FIGS. 2A, 2D, and 5A). APOBEC-1 conjugated to CMPK was used in this study because it showed a less robust editing activity in vitro and targeted primarily cytoplasmic mRNAs (Yang et al., “Induction of cytidine to uridine editing on cytoplasmic apolipoprotein B mRNA by overexpressing APOBEC-1,” J. Biol. Chem. 275:22663-22669 (2000), which is hereby incorporated by reference in its entirety). In vitro studies demonstrated that APOBEC-1 retained catalytic activity when conjugated to various lengths of non-specific proteins (Siddiqui et al., “Disproportionate relationship between APOBEC-1 expression and apoB mRNA editing activity,” Exp. Cell Res. 252:154-164 (1999); Yang et al., “Induction of cytidine to uridine editing on cytoplasmic apolipoprotein B mRNA by overexpressing APOBEC-1,” J. Biol. Chem. 275:22663-22669 (2000), each of which is hereby incorporated by reference in its entirety).

A double-stranded oligomeric nucleotide encoding the 9-amino acid TAT domain flanked by glycine residues (sense strand shown below, SEQ ID No: 25)

catatgggaa gaaaaaaaag aagacaaaga agaagaggcc tcgag 45

and a PCR product encoding HA-rAPOBEC-CMPK (SEQ ID No: 26 as set forth below)

| | |
|---|---|
| atgggctcta gataccccta cgacgtgccc gactacgccg atatcagttc cgagacaggc | 60 |
| cctgtagctg ttgatcccac tctgaggaga agaattgagc cccacgagtt tgaagtcttc | 120 |
| tttgaccccc gggaacttcg gaaagagacc tgtctgctgt atgagatcaa ctggggagga | 180 |
| aggcacagca tctggcgaca cacgagccaa aacaccaaca aacacgttga agtcaatttc | 240 |
| atagaaaaat ttactacaga aagatacttt tgtccaaaca ccagatgctc cattacctgg | 300 |
| ttcctgtcct ggagtccctg tggggagtgc tccagggcca ttacagaatt tttgagccga | 360 |
| tacccccatg taactctgtt tatttatata gcacggcttt atcaccacgc agatcctcga | 420 |
| aatcggcaag gactcassga ccttattagc agcggtgtta ctatccagat catgacggag | 480 |
| caagagtctg gctactgctg gaggaatttt gtcaactact ccccttcgaa tgaagctcat | 540 |
| tggccaaggt acccccatct gtgggtgagg ctgtacgtac tggaactcta ctgcatcatt | 600 |
| ttaggacttc caccctgttt aaatatttta agaagaaaac aacctcaact cacgtttttc | 660 |
| acgattgctc ttcaaagctg ccattaccaa aggctaccac cccacatcct gtgggccaca | 720 |
| gggttgaaag aattccacgc tgccatggca gacacctttc tggagcacat gtgccgcctg | 780 |
| gacatcgact ccgagccaac cattgccaga aacaccggca tcatctgcac catcggccca | 840 |
| gcctcccgct ctgtggacaa gctgaaggaa atgattaaat ctggaatgaa tgttgcccgc | 900 |
| ctcaacttct cgcacggcac ccacgagtat catgagggca caattaagaa cgtgcgagag | 960 |
| gccacagaga gctttgcctc tgacccgatc acctacagac ctgtggctat tgcactggac | 1020 |
| accaagggac ctgaaatccg aactggactc atcaagggaa gtggcacagc agaggtggag | 1080 |
| ctcaagaagg gcgcagctct caaagtgacg ctggacaatg ccttcatgga gaactgcgat | 1140 |
| gagaatgtgc tgtgggtgga ctacaagaac ctcatcaaag ttatagatgt gggcagcaaa | 1200 |
| atctatgtgg atgacggtct catttccttg ctggttaagg agaaaggcaa ggactttgtc | 1260 |
| atgactgagg ttgagaacgg tggcatgctt ggtagtaaga agggagtgaa cctcccaggt | 1320 |
| gctgcggtcg acctgcctgc agtctcagag aaggacattc aggacctgaa atttggcgtg | 1380 |
| gagcagaatg tggacatggt gttcgcttcc ttcatccgca aagctgctga tgtccatgct | 1440 |
| gtcaggaagg tgctagggga aaagggaaag cacatcaaga ttatcagcaa gattgagaat | 1500 |
| cacgagggtg tgcgcaggtt tgatgagatc atggaggcca gcgatggcat tatggtggcc | 1560 |
| cgtggtgacc tgggtattga gatccctgct gaaaaagtct tcctcgcaca gaagatgatg | 1620 |
| attgggcgct gcaacagggc tggcaaaccc atcatttgtg ccactcagat gttggaaagc | 1680 |
| atgatcaaga aacctcgccc gacccgcgct gagggcagtg atgttgccaa tgcagttctg | 1740 |
| gatggagcag actgcatcat gctgtctggg gagaccgcca agggagacta cccactggag | 1800 |
| gctgtgcgca tgcagcacgc tattgctcgt gaggctgagg ccgcaatgtt ccatcgtcag | 1860 |
| cagtttgaag aaatcttacg ccacagtgta caccacaggg agcctgctga tgccatggca | 1920 |
| gcaggcgcgg tggaggcctc ctttaagtgc ttagcagcag ctctgatagt tatgaccgag | 1980 |
| tctggcaggt ctgcacacct ggtgtcccgg taccgcccgc gggctcccat catcgccgtc | 2040 |
| acccgcaatg accaaacagc acgccaggca cacctgtacc gcggcgtctt ccccgtgctg | 2100 |
| tgcaagcagc cggcccacga tgcctgggca gaggatgtgg atctccgtgt gaacctgggc | 2160 |
| atgaatgtcg gcaaagcccg tggattcttc aagaccgggg acctggtgat cgtgctgacg | 2220 |
| ggctggcgcc ccggctccgg ctacaccaac accatgcggg tggtgcccgt gcca 2274 | |

or HA-CMPK (SEQ ID No: 27 as set forth below)

| | |
|---|---|
| ctcgagatgt acccctacga cgtgcccgac tacgccgata tccacgctgc catggcagac | 60 |
| acctttctgg agcacatgtg ccgcctggac atcgactccg agccaaccat tgccagaaac | 120 |
| accggcatca tctgcaccat cggcccagcc tcccgctctg tggacaagct gaaggaaatg | 180 |
| attaaatctg gaatgaatgt tgcccgcctc aacttctcgc acggcaccca cgagtatcat | 240 |
| gagggcacaa ttaagaacgt gcgagaggcc acagagagct ttgcctctga cccgatcacc | 300 |
| tacagacctg tggctattgc actggacacc aagggacctg aaatccgaac tggactcatc | 360 |
| aagggaagtg gcacagcaga ggtggagctc aagaagggcg cagctctcaa agtgacgctg | 420 |
| gacaatgcct tcatggagaa ctgcgatgag aatgtgctgt gggtggacta caagaacctc | 480 |
| atcaaagtta tagatgtggg cagcaaaatc tatgtggatg acggtctcat ttccttgctg | 540 |
| gttaaggaga aaggcaagga ctttgtcatg actgaggttg agaacggtgg catgcttggt | 600 |
| agtaagaagg gagtgaacct cccaggtgct gcggtcgacc tgcctgcagt ctcagagaag | 660 |
| gacattcagg acctgaaatt tggcgtggag cagaatgtgg acatggtgtt cgcttccttc | 720 |
| atccgcaaag ctgctgatgt ccatgctgtc aggaaggtgc taggggaaaa gggaaagcac | 780 |
| atcaagatta tcagcaagat tgagaatcac gagggtgtgc gcaggtttga tgagatcatg | 840 |
| gaggccagcg atggcattat ggtggcccgt ggtgacctgg gtattgagat ccctgctgaa | 900 |
| aaagtcttcc tcgcacagaa gatgatgatt gggcgctgca acagggctgg caaacccatc | 960 |
| atttgtgcca ctcagatgtt ggaaagcatg atcaagaaac ctcgcccgac ccgcgctgag | 1020 |
| ggcagtgatg ttgccaatgc agttctggat ggagcagact gcatcatgct gtctggggag | 1080 |
| accgccaagg gagactaccc actggaggct gtgcgcatgc agcacgctat tgctcgtgag | 1140 |
| gctgaggccg caatgttcca tcgtcagcag tttgaagaaa tcttacgcca cagtgtacac | 1200 |
| cacagggagc ctgctgatgc catggcagca ggcgcggtgg aggcctcctt taagtgctta | 1260 |
| gcagcagctc tgatagttat gaccgagtct ggcaggtctg cacacctggt gtcccggtac | 1320 |
| cgcccgcggg ctcccatcat cgccgtcacc cgcaatgacc aaacagcacg ccaggcacac | 1380 |
| ctgtaccgcg gcgtcttccc cgtgctgtgc aagcagccgg cccacgatgc ctgggcagag | 1440 |
| gatgtggatc tccgtgtgaa cctgggcatg aatgtcggca aagcccgtgg attcttcaag | 1500 |
| accggggacc tggtgatcgt gctgacgggc tggcgccccg gctccggcta caccaacacc | 1560 |
| atgcgggtgg tgcccgtgcc atgactcgag 1590 | |

(Yang et al., “Induction of cytidine to uridine editing on cytoplasmic apolipoprotein B mRNA by overexpressing APOBEC-1, ” J. Biol. Chem. 275:22663-22669 (2000), which is hereby incorporated by reference in its entirety) were inserted into Ndel/Xhol digested pPROEX vector (Life, Gaithersburg, Md.). The entire constructs (TAT-rAPOBEC -CMPK (SEQ ID No: 3) or TAT-CMPK (SEQ ID No: 28 as set forth below)

| | |
|---|---|
| catatgggaa gaaaaaaaag aagacaaaga agaagaggcc tcgagatgta cccctacgac | 60 |
| gtgcccgact acgccgatai ccacgctgcc atggcagaca cctttctgga gcacatgtgc | 120 |
| cgcctggaca tcgactccga gccaaccatt gccagaaaca ccggcatcat ctgcaccatc | 180 |
| ggcccagcct cccgctctgt ggacaagctg aaggaaatga ttaaatctgg aatgaatgtt | 240 |
| gcccgcctca acttctcgca cggcacccac gagtatcatg agggcacaat taagaacgtg | 300 |
| cgagaggcca cagagagctt tgcctctgac ccgatcacct acagacctgt ggctattgca | 360 |
| ctggacacca agggacctga aatccgaact ggactcatca agggaagtgg cacagcagag | 420 |
| gtggagctca agaagggcgc agctetcaaa gtgacgctgg acaatgcctt catggagaac | 480 |
| tgcgatgaga atgtgctgtg ggtggactac aagaacctca tcaaagttat agatgtgggc | 540 |
| agcaaaatct atgtggatga cggtctcatt tccttgctgg ttaaggagaa aggcaaggac | 600 |
| tttgtcatga ctgaggttga gaacggtggc atgcttggta gtaagaaggg agtgaacctc | 660 |
| ccaggtgctg cggtcgacct gcctgcagtc tcagagaagg acattcagga cctgaaattt | 720 |
| ggcgtggagc agaatgtgga catggtgttc gcttccttca tccgcaaagc tgctgatgtc | 780 |
| catgctgtca ggaaggtgct aggggaaaag ggaaagcaca tcaagattat cagcaagatt | 840 |
| gagaatcacg agggtgtgcg caggtttgat gagatcatgg aggccagcga tggcattatg | 900 |
| gtggcccgtg gtgacctggg tattgagatc cctgctgaaa aagtcttcct cgcacagaag | 960 |
| atgatgattg ggcgctgcaa cagggctggc aaacccatca tttgtgccac tcagatgttg | 1020 |
| gaaagcatga tcaagaaacc tcgcccgacc cgcgctgagg gcagtgatgt tgccaatgca | 1080 |
| gttctggatg gagcagactg catcatgctg tctggggaga ccgccaaggg agactaccca | 1140 |
| ctggaggctg tgcgcatgca gcacgctatt gctcgtgagg ctgaggccgc aatgttccat | 1200 |
| cgtcagcagt ttgaagaaat cttacgccac agtgtacacc acagggagcc tgctgatgcc | 1260 |
| atggcagcag gcgcggtgga ggcctccttt aagtgcttag cagcagctct gatagttatg | 1320 |
| accgagtctg gcaggtctgc acacctggtg tcccggtacc gcccgcgggc tcccatcatc | 1380 |
| gocgtcaccc gcaatgacca aacagcacgc caggcacacc tgtaccgcgg cgtcttcccc | 1440 |
| gtgctgtgca agcagccggc ccacgatgcc tgggcagagg atgtggatct ccgtgtgaac | 1500 |
| ctgggcatga atgtcggcaa agcccgtgga ttcttcaaga ccggggacct ggtgatcgtg | 1560 |
| ctgacgggct ggcgccccgg ctccggctac accaacacca tgcgggtggt gcccgtgcca | 1620 |
| tgact cgag 1629 | |

were inserted into pET-24b (Novagen, Madison, Wis.) vector to take advantage of the C-terminal His₆ tag. TAT fusion proteins (referred to as TAT-CMPK, the expression product of SEQ ID No: 28, and TAT-rAPOBEC-CMPK, SEQ ID No: 4) were purified from BL-21(DE3) codon plus cells (Stratagene, La Jolla, Calif.). Two to four 1-liter cultures were inoculated with a 10 ml overnight culture each and induced by 0.1 mM IPTG at 30° C. for 1 hour. Soluble proteins were obtained by French press in 25 ml of buffer A (8M urea, 10 mM Tris pH 8, 100 mM NaH₂PO₄). Cellular lysates were cleared by centrifugation, loaded onto a 5-ml Ni-NTA column (Qiagen, Valencia, Calif.) in buffer A with 10-20 mM imidazole, washed and eluted with imidazole in buffer A ‘stepwise’ (100, 175 and 250 mM) and loaded onto a HiTrap SP column (Amersham Pharmacia, Piscataway, N.J.). The column was washed and eluted with 1 M NaCl in buffer A. The urea and high salt were removed from the relevant fractions by rapid dialysis against buffer B (30 mM Tris pH=8.5, 50 mM NaCl, 10 μM zinc acetate, 5% glycerol). The elution profile was analyzed by SDS-PAGE. Gels were stained with silver according to manufacture's recommendations (Bio-Rad, Hercules, Calif.).

Recombinant proteins were solubilized in 8M urea buffer from bacterial cells so as to maximize their yield from inclusion bodies. Previous studies have shown that denatured proteins could transduce as well as native proteins (Schwarze et al., “In vivo protein transduction: delivery of a biologically active protein into the mouse,” Science 285:1569-1572 (1999), which is hereby incorporated by reference in its entirety). The proteins were purified through metal-chelating affinity chromatography followed by cationic exchange chromatography. The urea was removed by rapid dialysis and the purity of full-length 86 kDa TAT-rAPOBEC-CMPK, SEQ ID No: 4, was apparent as shown by silver staining (FIG. 5B). The purification of full-length protein was also confirmed by western blot using anti-His₆ antibody.

### Example 2

### In vitro Introduction of TAT-rAPOBEC-CMPK into McArdle Cells

The uptake of TAT-rAPOBEC-CMPK, SEQ ID No: 4, into McArdle cells was evaluated using an antibody reactive with the HA epitope and fluorescence microscopy.

McArdle RH7777 cells were obtained from ATCC (Manassas, Va.) and cultured as described previously (Yang et al., “Partial characterization of the auxiliary factors involved in apo B mRNA editing through APOBEC-1 affinity chromatography,” J. Biol. Chem 272:27700-27706 (1997), which is hereby incorporated by reference in its entirety). McArdle cells, grown on six well cluster plates were treated with either TAT-rAPOBEC-CMPK or TAT-CMPK for the indicated times. Cells were then washed extensively with PBS and subsequently fixed with 2% paraformaldehyde, permeabilized with 0.4% Triton X100, blocked with 1% BSA and reacted with affinity purified anti-HA (Babco, Berkeley, Calif.) and affinity purified FITC conjugated goat anti-mouse secondary antibody (Organon Teknika, West Chester, Pa.), each at 1:1000 dilution. Fluorescence was observed and electronic images captured on an inverted, fluorescence Olympus microscope.

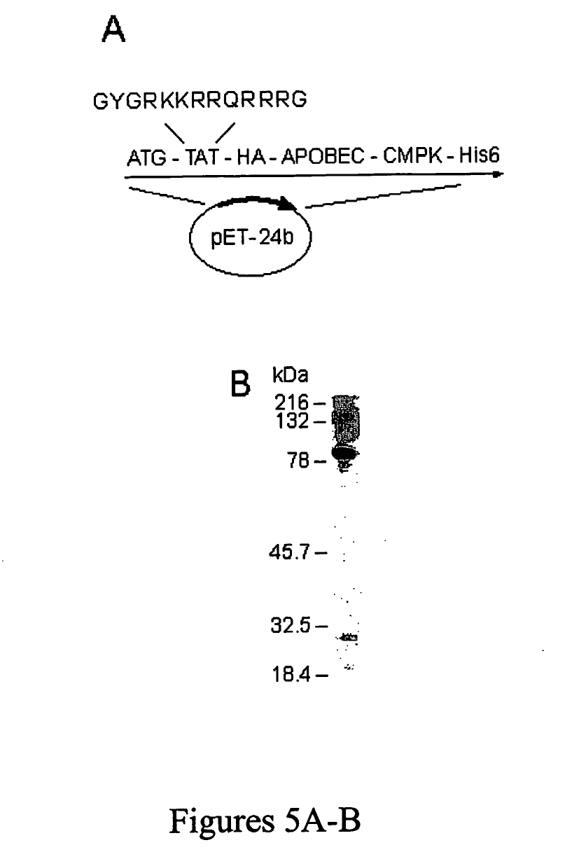

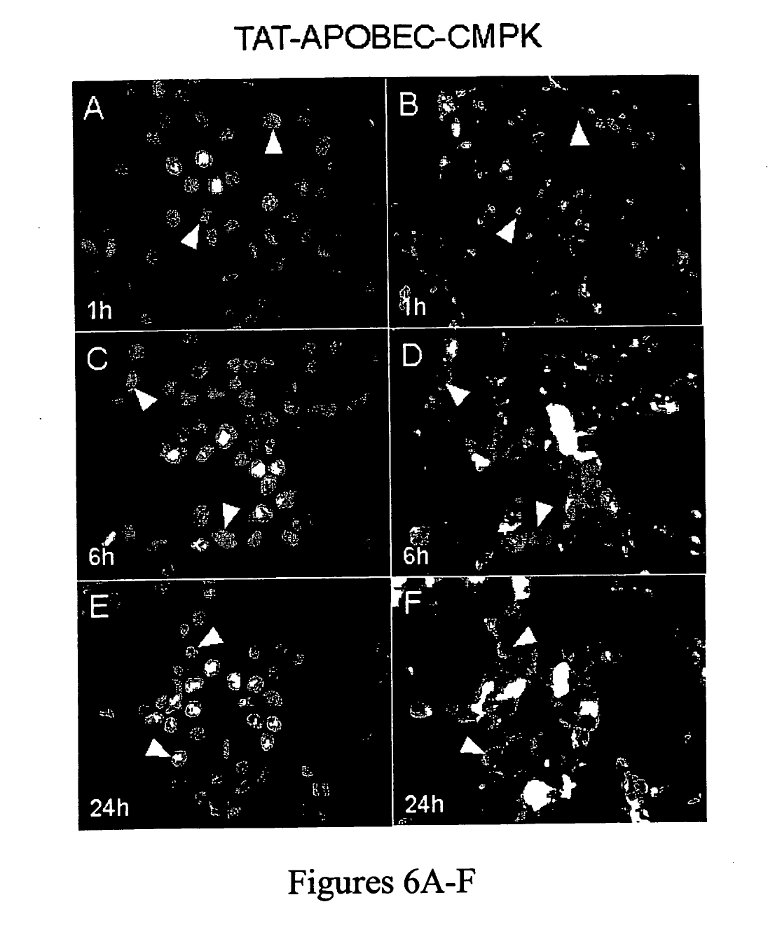

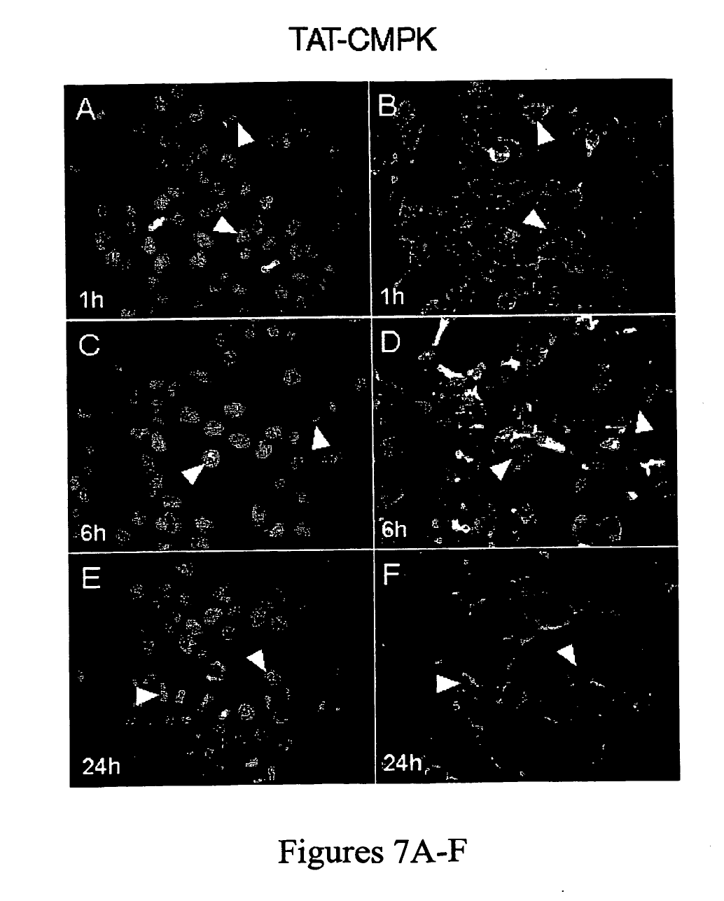

Recombinant APOBEC-1 has a tendency to aggregate, a property which persists in TAT-rAPOBEC-CMPK, apparent as aggregates of HA antibody-reactive material attached to the surface of cells 1 h following the addition of the protein to the media (FIGS. 6A-B). Aggregation was not a property of the TAT motif or CMPK as control protein (TAT-CMPK) at a higher molar concentration appeared as an array of speckles attached to the surface of McArdle cells 1 h following its addition to the media (FIGS. 7A and B).

Within 6 h following treatment, both TAT-rAPOBEC-CMPK (FIGS. 6C-D) and TAT-CMPK (FIGS. 7C-D) were apparent inside the cells and the cell surface-attached aggregates appeared to be more disperse. Following 24 h of treatment, many of the cells treated with TAT-rAPOBEC-CMPK demonstrated bright perinuclear fluorescence and also a low intensity of fluorescence throughout the nucleus and cytoplasm (FIGS. 6E-F). Cells treated for 24 h with TAT-CMPK demonstrated bright fluorescent speckles in the cytoplasm and fainter homogenous nuclear fluorescence (FIG. 7E-F). The nuclear distribution of the recombinant protein might have been facilitated by the embedded nuclear localization signal (NLS) in TAT sequence (Schwarze et al., “In vivo protein transduction: delivery of a biologically active protein into the mouse,” Science 285:1569-1572 (1999), which is hereby incorporated by reference in its entirety) as APOBEC-1 alone does not have a functional NLS (Yang et al., “Multiple protein domains determine the cell type-specific nuclear distribution of the catalytic subunit required for apo B mRNA editing,” Proc. Natl. Acad. Sci. USA 94:13075-13080 (1997), which is hereby incorporated by reference in its entirety) and 6His-HA-APOBEC-CMPK was excluded from the nucleus (Yang et al., “Induction of cytidine to uridine editing on cytoplasmic apolipoprotein B mRNA by overexpressing APOBEC-1,” J. Biol. Chem. 275:22663-22669 22669 (2000), which is hereby incorporated by reference in its entirety). The data suggested that both TAT-rAPOBEC-CMPK and TAT-CMPK were taken up by McArdle cells. Comparatively, the efficiency of TAT-rAPOBEC-CMPK uptake was poorer than that for TAT-CMPK, and the distribution of these proteins within the cells appeared different.

### Example 3

### Measurement of Apolipoprotein B mRNA Editing in TAT-rAPOBEC-CMPK Transduced McArdle Cells

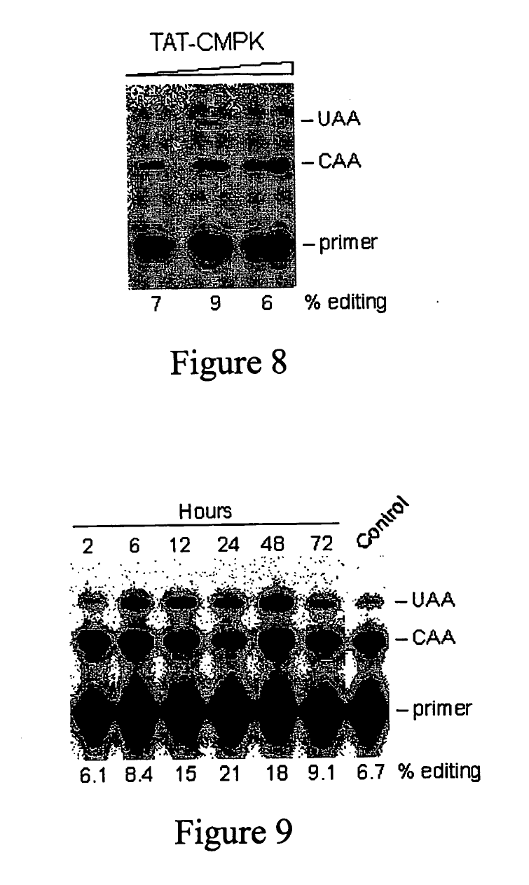

Given that TAT-CMPK entered McArdle cells, as demonstrated in Example 2, an evaluation was made as to whether this would affect apolipoprotein B mRNA editing activity (FIG. 8). Cells were treated with the indicated amounts of TAT-CMPK (using the same preparation of protein as in FIG. 7) and total cellular RNA was isolated following 24 h and the proportion of edited apolipoprotein B mRNA measured.

Total cellular RNA was isolated from cells with Tri-Reagent (Molecular Research Center, Cincinnati, Ohio) according to manufacture's recommendations. Purified RNAs were digested with RQ-DNase I (Promega, Madison, Wisc.) and with RsaI (Promega) restriction enzyme that has a recognition site between the PCR annealing sites of target substrates to ensure the removal of the contaminating genomic DNA.

Editing activity was determined by the reverse transcriptase-polymerase chain reaction (RT-PCR) methodology described previously (Smith et al. “In vitro apolipoprotein B mRNA editing: Identification of a 27S editing complex,” Proc. Natl. Acad. Sci. USA 88:1489-1493 (1991), which is hereby incorporated by reference in its entirety). First strand cDNA was generated using oligo dT-primed total cellular RNA. Specific PCR amplification of rat apolipoprotein B sequence surrounding the editing site was accomplished using ND1/ND2 primer pairs set forth below:

| ND1 | | |
|---|---|---|
| atctgactgg gagagacaag tag | 23 | (SEQ ID No: 29) |
| ND2 | | |
| gttcttttta agtcctgtgc atc | 23 | (SEQ ID No: 30) |

PCR products were gel isolated and the editing efficiency was determined by poisoned primer extension assay using ³²P ATP (NEN, Boston, Mass.) end-labeled DD3 primer (SEQ ID No: 31) as follows: aatcatgtaa atcataacta tctttaatat actga 35 under high concentration of dideoxy GTP as described previously (Smith et al. “In vitro apolipoprotein B mRNA editing: Identification of a 27S editing complex,” Proc. Natl. Acad. Sci. USA 88:1489-1493 (1991); Sowden et al., “Overexpression of APOBEC-1 results in mooring-sequence-dependent promiscuous RNA editing,” J. Biol. Chem. 271:3011-3017 (1996), each of which is hereby incorporated by reference in its entirety). Primer extension products were resolved on a 10% denaturing polyacrylamide gel, autoradiographed, and then quantified by a laser densitometric scanning (Molecular Dynamics, Sunnyvale, Calif.). Percent editing was calculated as the counts in the UAA (edited) band divided by the sum of the counts in UAA and those in the CAA (unedited) bands and multiplied by 100.

No change in the percent editing of apolipoprotein B mRNA relative to untreated cells (see FIG. 9) was observed with TAT-CMPK concentrations ranging from 45 to 1125 nM (5 to 133 μg protein/ml of media) (FIG. 8).

In contrast, editing activity increased in McArdle cells with 360 nM (62 μg protein/ml media) TAT-rAPOBEC-CMPK following 6 h and continued to a peak by 24 h, a more than 3-fold increase over the level of editing observed in control cells (FIG. 9). The proportion of edited RNA remained elevated up to 48 h after treatment (FIG. 9) and approached baseline by 72 h. It has been reported that the enzymatic activity lagged the appearance of the transduced protein inside the cells, probably due to a slow refolding of the transduced protein (Schwarze et al., “In vivo protein transduction: delivery of a biologically active protein into the mouse,” Science 285:1569-1572 (1999), which is hereby incorporated by reference in its entirety). Taken together, the results demonstrated that TAT-rAPOBEC-CMPK transduced into McArdle cells, refolded into an enzymatically active conformation over the first 6 hr and then edited apolipoprotein B mRNA. The reduction in the proportion of edited apolipoprotein B mRNA after 48 hr was likely due to enzyme inactivation and apolipoprotein B mRNA turnover. This characteristic was important as it demonstrated the transient and reversible nature of the protein transduction system.

### Example4

### In vitro Introduction of TAT-rAPOBEC-CMPK into Primary Hepatocytes

To determine if the results obtained using McArdle cells would be applicable in primary liver cells, cultured rat primary hepatocytes were prepared and then treated with TAT-rAPOBEC-CMPK. The rat primary hepatocytes were prepared from unfasted, male Sprague-Dawley rats (250-275 g body weight, Taconic Farm) fed ad libitum normal rat chow as described previously (Van Mater et al., “Ethanol increases apolipoprotein B mRNA editing in rat primary hepatocytes and McArdle cells,” Biochem. Biophys. Res. Comm. 252:334-339 (1998), which is hereby incorporated by reference in its entirety). Recombinant TAT fusion protein was added directly to the cell culture media after dialysis.

It has been shown that the editing efficiency in primary rat hepatocytes decreased as a result of proliferation after 72 hours in culture (Van Mater et al., “Ethanol increases apolipoprotein B mRNA editing in rat primary hepatocytes and McArdle cells,” Biochem. Biophys. Res. Comm. 252:334-339 (1998), which is hereby incorporated by reference in its entirety). Together with the fact that TAT-rAPOBEC-CMPK maximally increased editing 24 hours after treatment in McArdle cells, a decision was made to evaluate dose response for a fixed time rather than study kinetics. Primary hepatocytes were treated with the indicated amounts of TAT-rAPOBEC-CMPK and analyzed for edited apolipoprotein B mRNA 24 hours afterwards. Analysis of apolipoprotein B mRNA was carried out a described in Example 3 above.

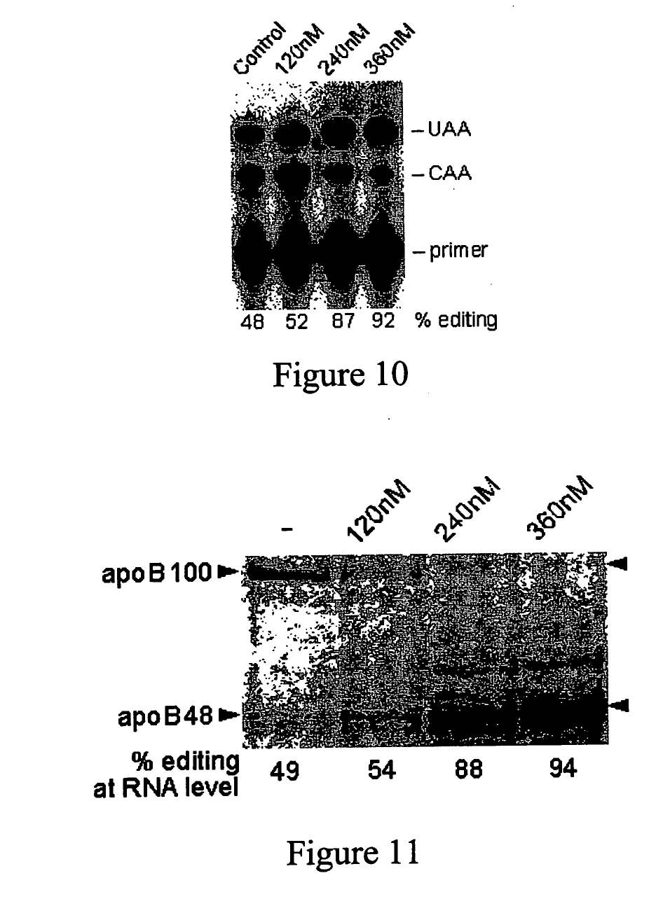

The editing activity of hepatocytes increased in proportion to the amount of TAT-rAPOBEC-CMPK added to the cell culture media relative to cells treated with buffer alone (FIG. 10) or treated with TAT-CMPK (FIG. 8). Given that the primary hepatocytes were seeded at the same cell number as McArdle cells, a comparison of the data in FIGS. 9 and 10 suggested that TAT-rAPOBEC-CMPK was more effective in inducing editing activity in the primary cell culture. This was true for several preparations of recombinant protein and primary cells and, therefore, the difference may be due to the fact that the primary hepatocytes have a higher baseline of editing than McArdle cells (48% versus 7%) and/or may be “primed” with more auxiliary factors.

Promiscuous editing of additional cytidines in rat apolipoprotein B mRNA of transfected cells (Sowden et al., “Overexpression of APOBEC-1 results in mooring-sequence-dependent promiscuous RNA editing,” J. Biol. Chem. 271:3011-3017 3017 (1996); Yamanaka et al., “Hyperediting of multiple cytidines of apolipoprotein B mRNA by APOBEC-1 requires auxiliary protein(s) but not a mooring sequence motif,” J. Biol. Chem. 271:11506-11510 (1996); Sowden et al., “Apolipoprotein B RNA Sequence 3′ of the mooring sequence and cellular sources of auxiliary factors determine the location and extent of promiscuous editing,” Nucleic Acids Res. 26:1644-1652 (1998), each of which is hereby incorporated by reference in its entirety) or hyper-editing of other mRNAs in transgenic mice and rabbits (Yamanaka et al., “Hyperediting of multiple cytidines of apolipoprotein B mRNA by APOBEC-1 requires auxiliary protein(s) but not a mooring sequence motif,” J. Biol. Chem. 271:11506-11510 (1996); Yamanaka et al., “A novel translational repressor mRNA is edited extensively in livers containing tumors caused by the transgene expression of the apoB mRNA editing enzyme,” Genes & Dev. 11:321-333 (1997), each of which is hereby incorporated by reference in its entirety) has been observed in response to very high levels of APOBEC-1 expression. Editing of cytidines 5′ of the wild type editing site (C6666) was a bellwether for the loss of editing site fidelity in rat cells and could be used to monitor the induction of promiscuous editing in relation to changes in APOBEC-1 expression (Sowden et al., “Apolipoprotein B RNA Sequence 3′ of the mooring sequence and cellular sources of auxiliary factors determine the location and extent of promiscuous editing,” Nucleic Acids Res. 26:1644-1652 (1998); Siddiqui et al., “Disproportionate relationship between APOBEC-1 expression and apoB mRNA editing activity,” Exp. Cell Res. 252:154-164 (1999), each of which is hereby incorporated by reference in its entirety). Promiscuous editing of cytidine 3′ C6666 in apolipoprotein B mRNA did not occur to a significant extent in rat cells and hyperediting of mRNAs other than apolipoprotein B was not a characteristic of APOBEC-1 overexpression in rat cells (Sowden et al., “Apolipoprotein B RNA Sequence 3′ of the mooring sequence and cellular sources of auxiliary factors determine the location and extent of promiscuous editing,” Nucleic Acids Res. 26:1644-1652 (1998), which is hereby incorporated by reference in its entirety).

Despite the high level of editing activity in treated primary hepatocytes, promiscuous editing (evident as additional primer extension products above UAA (Sowden et al., “Determinants involved in regulating the proportion of edited apolipoprotein B RNAs,” RNA 2:274-288 (1996); Sowden et al., “Apolipoprotein B RNA Sequence 3′ of the mooring sequence and cellular sources of auxiliary factors determine the location and extent of promiscuous editing,” Nucleic Acids Res. 26:1644-1652 (1998), each of which is hereby incorporated by reference in its entirety) was not observed (FIG. 10). Given that our detection limit for promiscuous editing was 0.3% (Sowden et al., “Determinants involved in regulating the proportion of edited apolipoprotein B RNAs,” RNA 2:274-288 (1996), which is hereby incorporated by reference in its entirety) the data suggested that TAT-rAPOBEC-CMPK could be used to substantially increase site-specific editing of apolipoprotein B mRNA without significant loss of fidelity of the reaction.

### Example 5

### Analysis of Secreted Lipoprotein Products by Transduced Primary Hepatocytes

To further confirm the efficacy of this method, secreted apolipoprotein B protein was evaluated in primary rat hepatocytes that were long-term metabolically labeled with [³⁵S]-methionine and [³⁵S]-cysteine after TAT-rAPOBEC-CMPK treatment.

Twelve to eighteen hour rat primary hepatocytes grown in Waymouth's 752/1 media (Sigma, St. Louis, Mo.) were treated for 11 hours with TAT-rAPOBEC-CMPK and then incubated for 1 hour in DMEM deficient medium (without methionine, cysteine and L-glutamine) (Sigma, St. Louis, Mo.) containing 0.2% (w/v) BSA, 0.1 nM insulin, 100 μg/ml streptomycin and 50 μg/ml gentamicin. The medium was replaced with fresh labeling medium containing 0.7 μCi/ml L-[³⁵S]-Methionine and L-[³⁵S]-Cysteine using EXPRE³⁵S³⁵S protein labeling mix (NEN, Boston, Mass.). Cells were incubated in the labeling medium for 30 minutes. One volume of Waymouth's medium with cold cysteine and methionine was added to cells and the labeling continued for an additional 12 hours, after which cell culture medium was collected for the isolation and analysis of secreted apolipoprotein B protein and RNAs. (RNA analysis was conducted as in Example 3 above.)

Immunoprecipitation of apolipoprotein B from cell culture medium was performed as described previously (Sparks et al., “Insulin-mediated inhibition of apolipoprotein B secretion requires an intracellular trafficking event and phosphatidylinositol 3-kinase activation: studies with brefeldin A and wortmannin in primary cultures of rat hepatocytes,” Biochem. J. 313:567-574 (1996), which is hereby incorporated by reference in its entirety). A rabbit polyclonal antibody raised against rat apolipoprotein B and reactive with the N-terminus of apolipoprotein B 100 and apolipoprotein B48 (obtained from Drs. J. D. Sparks and C. E. Sparks, University of Rochester) was used to precipitate apolipoprotein B. The immunoprecipitants were separated by SDS-PAGE on 5% gel. The gel was dried and exposed to film to reveal the secreted apolipoprotein B containing lipoprotein profile which represents the secreted apolipoprotein B48 and apolipoprotein B 100 during the 12 hour labeling period.

The secreted [³⁵S]-labeled apolipoprotein B lipoproteins were isolated from the cell culture media exposed to cells for 12 hours followed by immunoprecipitation, and analyzed by autoradiography after SDS-PAGE separation. The signal on the gel was in direct proportion to the number of cysteine and methionine residues in apolipoprotein B 100 and apolipoprotein B48. Since apolipoprotein B48 was the N-terminal 48% of apolipoprotein B 100, stronger signal was expected from apolipoprotein B 100 in control cells. However, as the editing efficiency approached 90% due to TAT-rAPOBEC-CMPK treatment, an increasing amount of apolipoprotein B48 was secreted, and apolipoprotein B 100 became almost undetectable (FIG. 11). Thus, lowering apolipoprotein B100 associated atherogenic risk factors through precisely controlled hepatic apolipoprotein B mRNA editing was achievable by protein transduction with TAT-rAPOBEC-CMPK.

Discussion of Examples 1-5

It is believed that the present invention offers a novel approach to curtail hepatic output of apolipoprotein B 100 associated atherogenic factors through up-regulating apolipoprotein B mRNA editing by using protein transduction into target (e.g., liver) cells. The PTD, amino acid residues 49-57, of HIV-1 TAT protein has been used in other systems to deliver functional full-length protein molecules into cells (Nagahara et al., “Transduction of full-length TAT fusion proteins into mammalian cells: TAT-p27^{Kip1} induces cell migration,” Nature Med. 4:1449-1452 (1998); Schwarze et al., “In vivo protein transduction: delivery of a biologically active protein into the mouse,” Science 285:1569-1572 (1999); Vocero-Akbani et al., “Killing HIV-infected cells by transduction with an HIV protease-activated caspase-3 protein,” Nature Med. 5:29-33 (1999), each of which is hereby incorporated by reference in its entirety). Some of these fusion molecules, when introduced into mice, entered all tissue cells, even crossing the blood brain barrier (Schwarze et al., “In vivo protein transduction: delivery of a biologically active protein into the mouse,” Science 285:1569-1572 (1999), which is hereby incorporated by reference in its entirety). Although the detailed mechanism for the cellular uptake of the fusions remains unknown, denaturing of the protein during membrane transduction is thought to be a rapid process and the rate limiting event is the renaturing of the transduced protein once inside of cells (Schwarze et al., “Protein transduction: unrestricted delivery into all cells,” Trends Cell Biol. 10:290-295 (2000), which is hereby incorporated by reference in its entirety).

In this regard, the protein transduction method may have limitations in that some proteins may not be able successfully to adopt an active conformation after they have been unfolded. It is significant, therefore, that the above Examples demonstrate that both TAT-CMPK (expression product of SEQ ID No: 28) and TAT-rAPOBEC-CMPK (SEQ ID No: 4) had the capacity to enter hepatyocytes and that TAT-rAPOBEC-CMPK activated editing within 6 hours of its addition to the media. Similar kinetics have been observed with TAT-rAPOBEC-CMPK prepared under native conditions.

Importantly, TAT-CMPK could not stimulate editing activity, demonstrating that the observed changes in editing were specific to APOBEC-1 containing recombinant proteins. Considering the tendency for APOBEC-1 containing proteins to aggregate, part of the lag in entering cells could have been due to the inability of these multimeric complexes to cross the plasma membrane and the time it took for TAT-rAPOBEC-CMPK monomers to dissociate from the aggregates and cross the membrane. This is supported by the finding that TAT-CMPK, which did not appear to form large aggregates, appeared to accumulate within the cells with more rapid kinetics than that observed for TAT-rAPOBEC-CMPK. The six hour lag before an increase in editing activity could be measured may have also been due to the time required for the transduced protein to refold and assemble editosomes.

Apolipoprotein B mRNA editing occurs in the cell nucleus despite the fact that editing factors can also be demonstrated in the cytoplasm (Yang et al., “Induction of cytidine to uridine editing on cytoplasmic apolipoprotein B mRNA by overexpressing APOBEC-1,” J. Biol. Chem. 275:22663-22669 (2000), which is hereby incorporated by reference in its entirety). The mechanism responsible for APOBEC-1's distribution in the nucleus is not understood (Yang et al., “Intracellular Trafficking Determinants in APOBEC-1, the Catalytic Subunit for Cytidine to Uridine Editing of ApoB mRNA,” Exp. Cell Res. 267:163-184 (2001), which is hereby incorporated by reference in its entirety), however its mass appeared to be important as the chimeric protein APOBEC-CMPK was excluded from the nucleus (Yang et al., “Multiple protein domains determine the cell type-specific nuclear distribution of the catalytic subunit required for apo B mRNA editing,” Proc. Natl. Acad. Sci. USA 94:13075-13080 (1997); Yang et al., “Induction of cytidine to uridine editing on cytoplasmic apolipoprotein B mRNA by overexpressing APOBEC-1,” J. Biol. Chem. 275:22663-22669 (2000), each of which is hereby incorporated by reference in its entirety). TAT-rAPOBEC -CMPK's ability to distribute in both the cytoplasm and the nucleus was consistent with the proposed ability of the TAT PTD to act also as a nuclear localization signal (Schwarze et al., “In vivo protein transduction: delivery of a biologically active protein into the mouse,” Science 285:1569-1572 (1999), which is hereby incorporated by reference in its entirety). Although TAT-rAPOBEC-CMPK's distribution mimicked that of the wild type enzyme's distribution (Yang et al., “Multiple protein domains determine the cell type-specific nuclear distribution of the catalytic subunit required for apo B mRNA editing,” Proc. Natl. Acad. Sci. USA 94:13075-13080 (1997), which is hereby incorporated by reference in its entirety), uncertainty remains as to whether all of the transduced TAT-rAPOBEC-CMPK molecules were active in editing, as well as whether cytoplasmic or nuclear transcripts were edited. Nonetheless, regardless of the degree of activity or its localization within the cell, a positive reduction in apolipoprotein B 100 lipoprotein was demonstrated.

Enhancement of editing activity by overexpression of APOBEC-1 through gene transfer has been shown to be associated with promiscuous editing on both nuclear and cytoplasmic transcripts (Sowden et al., “Overexpression of APOBEC-1 results in mooring-sequence-dependent promiscuous RNA editing,” J. Biol. Chem. 271:3011-3017 (1996); Yang et al., “Induction of cytidine to uridine editing on cytoplasmic apolipoprotein B mRNA by overexpressing APOBEC-1,” J. Biol. Chem. 275:22663-22669 (2000), each of which is hereby incorporated by reference in its entirety). Metabolic stimulation of apolipoprotein B mRNA editing always retained fidelity (Wu et al., “ApoB mRNA editing: validation of a sensitive assay and developmental biology of RNA editing in the rat,” J. Biol. Chem. 265:12312-12316 (1990); Greeve et al., “Apolipoprotein B mRNA editing in 12 different mammalian species: hepatic expression is reflected in low concentrations of apoB-containing plasma lipoproteins,” J. Lipid Res. 34:1367-1383 (1993); Phung et al., “Regulation of hepatic apoB RNA editing in the genetically obese Zucker rat,” Metabolism 45:1056-1058 (1996); von Wronski et al., “Insulin increases expression of apobec-1, the catalytic subunit of the apoB B mRNA editing complex in rat hepatocytes,” Metabolism Clinical & Exp. 7:869-873 (1998), each of which is hereby incorporated by reference in its entirety). It is highly significant, therefore, that the fidelity of the editing activity was retained with TAT-rAPOBEC-CMPK even when editing was enhanced to >90%. This level of high fidelity editing could not be achieved without hyper-editing in apobec-1 transgenic animals (Yamanaka et al., “Hyperediting of multiple cytidines of apolipoprotein B mRNA by APOBEC-1 requires auxiliary protein(s) but not a mooring sequence motif,” J. Biol. Chem. 271:11506-11510 (1996); Yamanaka et al., “A novel translational repressor mRNA is edited extensively in livers containing tumors caused by the transgene expression of the apoB mRNA editing enzyme,” Genes & Dev. 11:321-333 (1997); Sowden et al., “Overexpression of APOBEC-1 results in mooring-sequence-dependent promiscuous RNA editing,” J. Biol. Chem. 271:3011-3017 (1996); Sowden et al., “Apolipoprotein B RNA Sequence 3′ of the mooring sequence and cellular sources of auxiliary factors determine the location and extent of promiscuous editing,” Nucleic Acids Res. 26:1644-1652 (1998); each of which is hereby incorporated by reference in its entirety). There was no pathology in transgenic animals in which induction of hepatic apolipoprotein B mRNA editing was achieved at a low level of apobec-1 expression and these animals had a markedly lower serum apolipoprotein B 100 and significantly reduced serum LDL compared to controls (Teng et al., “Adenovirus-mediated gene transfer of rat apolipoprotein B mRNA editing protein in mice virtually eliminates apolipoprotein B-100 and normal low density lipoprotein production,” J. Biol. Chem. 269:29395-29404 (1994); Hughs et al., “Gene transfer of cytidine deaminase APOBEC-1 lowers lipoprotein(a) in transgenic mice and induces apolipoprotein B mRNA editing in rabbits,” Hum. Gene Ther. 7:39-49 (1996); Kozarsky et al., “Hepatic expression of the catalytic subunit of the apolipoprotein B mRNA editing enzyme ameliorates hypercholesterolemia in LDL receptor-deficient rabbits,” Hum. Gene Ther. 7:943-957 (1996); Farese et al., “Phenotypic analysis of mice expressing exclusively apolipoprotein B48 or apolipoprotein B 100,” Proc. Natl. Acad. Sci. USA 93:6393-6398 (1996); Qian et al., “Low expression of the apolipoprotein B mRNA editing transgene in mice reduces LDL but does not cause liver dysplasia or tumors,” Arteriosc. Thromb. Vasc. Biol. 18:1013-1020 (1998); Wu et al., “Normal perinatal rise in serum cholesterol is inhibited by hepatic delivery of adenoviral vector expressing apolipoprotein B mRNA editing enzyme in rabbits,” J. Surg. Res. 85:148-157 (1999), each of which is hereby incorporated by reference in its entirety). Interestingly, apobec-1 gene transfer into apobec-1 gene knockout mice restored editing and reduced serum LDL levels (Nakamuta et al., “Complete phenotypic characterization of the apobec-1 knockout mice with a wild-type genetic background and a human apolipoprotein B transgenic background, and restoration of apolipoprotein B mRNA editing by somatic gene transfer of Apobec-1,” J. Biol. Chem. 271:25981-25988 (1996), which is hereby incorporated by reference in its entirety), demonstrating that APOBEC-1 has therapeutic potential in livers with no prior editing activity. The induction of hepatic editing of apolipoprotein B mRNA in apobec-1 transgenic rabbits with an LDL receptor deficiency also ameliorated hypercholesterolemia (Kozarsky et al., “Hepatic expression of the catalytic subunit of the apolipoprotein B mRNA editing enzyme ameliorates hypercholesterolemia in LDL receptor-deficient rabbits,” Hum. Gene Ther. 7:943-957 (1996), which is hereby incorporated by reference in its entirety). Taken together, these studies suggested that apolipoprotein B mRNA editing could be safely targeted as a mechanism for reducing serum LDL and the risk of atherogenic diseases.

However, controlling a low level of apobec-1 expression using gene therapy is difficult and, quite often, unpredictable. For all of these reasons, despite the limited success of gene therapy approaches, gene therapy using apobec-1 does not appear to be a promising avenue which can be pursued for preventative or therapeutic control over atherogenic disease factors. The advantage of protein transduction therapy is that the dose can be modulated relative to the desired response and that the effect on editing can be terminated by withdrawing therapy.

The PTD should allow protein to enter all cells of the body, even if the protein is delivery intravenously (Schwarze et al., “In vivo protein transduction: delivery of a biologically active protein into the mouse,” Science 285:1569-1572 (1999), which is hereby incorporated by reference in its entirety). Ideally the liver should be specifically targeted with TAT-rAPOBEC-CMPK and an intraperitoneal injection can be utilized to accomplish a first pass clearance, transducing most of the protein into hepatocytes. Even though APOBEC-1 is not widely expressed in tissues (Teng et al., “Molecular cloning of an apo B messenger RNA editing protein,” Science 260:18116-1819 (1993), which is hereby incorporated by reference in its entirety), its generalized expression in transgenic animals did not induce pathology (Teng et al., “Adenovirus-mediated gene transfer of rat apolipoprotein B mRNA editing protein in mice virtually eliminates apolipoprotein B-100 and normal low density lipoprotein production,” J. Biol. Chem. 269:29395-29404 (1994); Hughs et al., “Gene transfer of cytidine deaminase APOBEC-1 lowers lipoprotein(a) in transgenic mice and induces apolipoprotein B mRNA editing in rabbits,” Hum. Gene Ther. 7:39-49 (1996); Kozarsky et al., “Hepatic expression of the catalytic subunit of the apolipoprotein B mRNA editing enzyme ameliorates hypercholesterolemia in LDL receptor-deficient rabbits,” Hum Gene Ther. 7:943-957 (1996); Farese et al., “Phenotypic analysis of mice expressing exclusively apolipoprotein B48 or apolipoprotein B 100, ” Proc. Natl. Acad. Sci. USA 93:6393-6398 (1996); Qian et al., “Low expression of the apolipoprotein B mRNA editing transgene in mice reduces LDL but does not cause liver dysplasia or tumors,” Arteriosc. Thromb. Vasc. Biol. 18:1013-1020 (1998); Wu et al., “Normal perinatal rise in serum cholesterol is inhibited by hepatic delivery of adenoviral vector expressing apolipoprotein B mRNA editing enzyme in rabbits,” J. Surg. Res. 85:148-157 (1999), each of which is hereby incorporated by reference in its entirety).

Uptake of TAT-rAPOBEC-CMPK or TAT-hAPOBEC-CMPK is unlikely to induce any side effects. Aside from one study suggesting that overexpression of APOBEC-1 in liver can lead to editing of mRNAs other than apolipoprotein B (Yamanaka et al., “A novel translational repressor mRNA is edited extensively in livers containing tumors caused by the transgene expression of the apoB mRNA editing enzyme,” Genes & Dev. 11:321-333 (1997), which is hereby incorporated by reference in its entirety) no other mRNA substrates for APOBEC-1 have been found (Skuse et al., Neurofibromatosis type I mRNA undergoes base-modification RNA editing,” Nucleic Acids Res. 24:478-486 (1996); Sowden et al., “Apolipoprotein B RNA Sequence 3′ of the mooring sequence and cellular sources of auxiliary factors determine the location and extent of promiscuous editing,” Nucleic Acids Res. 26:1644-1652 (1998), each of which is hereby incorporated by reference in its entirety). Furthermore, apobec-1 gene knock out studies have shown that there were no other editing enzymes capable of editing apolipoprotein B mRNA and that APOBEC-1 was not required for life (Hirano et al., “Targeted disruption of the mouse apobec-1 gene abolishes apolipoprotein B mRNA editing and eliminates apolipoprotein B48, ” J. Biol. Chem. 271:9887-9890 (1996); Nakamuta et al., “Complete phenotypic characterization of the apobec-1 knockout mice with a wild-type genetic background and a human apolipoprotein B transgenic background, and restoration of apolipoprotein B mRNA editing by somatic gene transfer of Apobec-1, ” J. Biol. Chem. 271:25981-25988 (1996), each of which is hereby incorporated by reference in its entirety). Taken together the data suggest that mRNA editing by APOBEC is self-limited due to its specificity for apolipoprotein B mRNA and, therefore, neither TAT-rAPOBEC-CMPK nor TAT-hAPOBEC-CMPK is likely to have effects in tissues others than those which express apolipoprotein B mRNA and auxiliary proteins.

Current cholesterol-lowering therapies target circulating cholesterol at the level of enhanced elimination or reduced production. A sector of the population remains at risk for atherosclerosis due to side effects from current therapies in some of these patients and the inability of others with defects in apolipoprotein B and/or the LDL receptor mediated uptake pathway to completely benefit from conventional cholesterol lowering therapies. Hypercholesterolemia is an early onset disease yet the restricted usage of conventional therapies among children due to the potential of interfering with pubertal development has not been resolved. Protein based therapies such as insulin or growth hormone have been extensively used among children to treat Type I diabetes or pituitary dwarfism, respectively. To the patient or the parent of the patient, the reversible nature of protein based therapy may be more appealing than gene therapy. To this end, the above results illustrate an alternative to conventional or gene therapy approaches for reducing the risk of atherosclerosis in the sectors of population at risk.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A chimeric protein comprising:
a first polypeptide comprising a protein transduction domain; and
a second polypeptide comprising APOBEC-1 or a fragment thereof which can edit mRNA encoding apolipoprotein B.

2. The chimeric protein according to claim 1 wherein the protein transduction domain is an HIV TAT protein transduction domain.

3. The chimeric protein according to claim 2, wherein the HIV TAT protein transduction domain comprises an amino acid sequence of SEQ ID No: 9.

4. The chimeric protein according to claim 1 wherein the APOBEC-1 or fragment thereof comprises an amino acid sequence of SEQ ID No: 11, SEQ ID No: 13, or SEQ ID No: 15, or fragments thereof.

5. The chimeric protein according to claim 1 further comprising:
a third polypeptide comprising a cytoplasmic localization protein or a fragment thereof which, upon cellular uptake of the chimeric protein, enhances localization of the chimeric protein to the cytoplasm.

6. The chimeric protein according to claim 5 wherein the cytoplasmic localization protein or fragment thereof is chicken muscle pyruvate kinase or a fragment thereof.

7. The chimeric protein according to claim 6 wherein the chicken muscle pyruvate kinase or a fragment thereof comprises an amino acid sequence of SEQ ID No: 17 or fragments thereof.

8. The chimeric protein according to claim 5 wherein, within the chimeric protein, the third polypeptide is C-terminal of the second polypeptide.

9. The chimeric protein according to claim 1 further comprising:
a third polypeptide comprising a plurality of adjacent histidine residues.

10. The chimeric protein according to claim 1 further comprising:
a third polypeptide comprising a hemagglutinin domain.

11. The chimeric protein according to claim 1 wherein, within the chimeric protein, the first polypeptide is N-terminal of the second polypeptide.

12. The chimeric protein according to claim 1, wherein the chimeric protein comprises an amino acid sequence of SEQ ID No: 2 or SEQ ID No: 4.

13. The chimeric protein according to claim 1, wherein the chimeric protein is in isolated form.

14. A composition comprising:
a pharmaceutically acceptable carrier and
the chimeric protein according to claim 1.

15. The composition according to claim 14, wherein the chimeric protein is present in an amount which is effective to modify apolipoprotein B mRNA editing in liver cells which uptake the chimeric protein.

16. The composition according to claim 14, wherein the composition is in the form of a tablet; capsule, powder, solution, suspension, or emulsion.

17. A chimeric protein comprising:
a first polypeptide comprising a protein transduction domain; and
a second polypeptide comprising ACF or a fragment thereof which can bind to apolipoprotein B mRNA to facilitate editing of the mRNA by APOBEC-1.

18. The chimeric protein according to claim 17 wherein the protein transduction domain is an HIV tat protein transduction domain.

19. The chimeric protein according to claim 18, wherein the HIV tat protein transduction domain comprises an amino acid sequence of SEQ ID No: 9.

20. The chimeric protein according to claim 17 wherein the ACF or fragment thereof comprises an amino acid sequence of SEQ ID No: 21 or SEQ ID No: 23 or fragments thereof.

21. The chimeric protein according to claim 17 further comprising:
a third polypeptide comprising a plurality of adjacent histidine residues.

22. The chimeric protein according to claim 17 further comprising:
a third polypeptide comprising a hemagglutinin domain.

23. The chimeric protein according to claim 17 wherein, within the chimeric protein, the first polypeptide is N-terminal of the second polypeptide.

24. The chimeric protein according to claim 17 wherein the chimeric protein comprises an amino acid sequence of SEQ ID No: 6 or SEQ ID No: 8.

25. The chimeric protein according to claim 17 wherein the chimeric protein is in isolated form.

26. A composition comprising:
a first chimeric protein comprising (i) a first polypeptide comprising a protein transduction domain and (ii) a second polypeptide comprising APOBEC-1 or a fragment thereof which can edit the mRNA encoding apolipoprotein B; and
a second chimeric protein comprising (i) a first polypeptide comprising a protein transduction domain and (ii) a second polypeptide comprising ACF or a fragment thereof which can bind to apolipoprotein B mRNA to facilitate editing of the mRNA by APOBEC-1 or the fragment thereof.

27. The composition according to claim 26 wherein
the first chimeric protein is present in an amount which is effective to modify apolipoprotein B mRNA editing in cells which uptake the first chimeric protein and
the second chimeric protein is present in an amount which is effective to bind apolipoprotein B mRNA and assist the first chimeric protein in modifying apolipoprotein B mRNA in cells which uptake the first and second chimeric proteins.

28. The composition according to claim 26 wherein the first chimeric protein comprises an amino acid sequence of SEQ ID No: 2 or SEQ ID No: 4.

29. The composition according to claim 26 wherein the second chimeric protein comprises an amino acid sequence of SEQ ID No: 6 or SEQ ID No: 8.

30. The composition according to claim 26 further comprising:
a pharmaceutically acceptable carrier in which the first and second chimeric proteins are dispersed.

31. The composition according to claim 26 wherein the composition is in the form of a tablet, capsule, powder, solution, suspension, or emulsion.

32. A DNA molecule encoding a chimeric protein according to claim 1.

33. The DNA molecule according to claim 32 comprising a nucleotide sequence of SEQ ID No: 1 or SEQ ID No: 3.

34. A DNA construct comprising:
the DNA molecule according to claim 32;
a promoter sequence operably connected 5′ to the DNA molecule; and
a 3′ regulatory sequence operably connected 3′ of the DNA molecule.

35. An expression vector comprising a DNA molecule according to claim 32.

36. A recombinant host cell transformed with a DNA molecule according to claim 32.

37. A DNA molecule encoding a chimeric protein according to claim 17.

38. The DNA molecule according to claim 37 comprising a nucleotide sequence of SEQ ID No: 5 or SEQ ID No: 7.

39. A DNA construct comprising:
the DNA molecule according to claim 37;
a promoter sequence operably connected 5′ to the DNA molecule; and
a 3′ regulatory sequence operably connected 3′ of the DNA molecule.

40. An expression vector comprising a DNA molecule according to claim 37.

41. A recombinant host cell transformed with a DNA molecule according to claim 37.

42. A delivery device comprising a chimeric protein according to claim 1.

43. The delivery device according to claim 42, wherein the delivery device is in the form of a liposome, a niosome, a transdermal patch, an implant, or a syringe.

44. A delivery device comprising a composition according to claim 14.

45. The delivery device according to claim 44, wherein the delivery device is in the form of a liposome, a niosome, a transdermal patch, an implant, or a syringe.

46. A delivery device comprising a composition according to claim 26.

47. The delivery device according to claim 46, wherein the delivery device is in the form of a liposome, a niosome, a transdermal patch, an implant, or a syringe.

48. A method of modifying apolipoprotein B mRNA editing in vivo comprising:
contacting apolipoprotein B mRNA in a cell with a chimeric protein according to claim 1 under conditions effective to increase the concentration of apolipoprotein B48 which is secreted by the cell as compared to the concentration of apolipoprotein B100 which is secreted by the cell, relative to an untreated cell.

49. The method according to claim 48 wherein the cell is a liver cell.

50. The method according to claim 48 wherein the cell is present in a mammal.

51. The method according to claim 48 further comprising prior to said contacting:
exposing the cell to the chimeric protein under conditions effective to induce cellular uptake of the chimeric protein.

52. The method according to claim 48 wherein the chimeric protein comprises an amino acid sequence of SEQ ID No: 2 or SEQ ID No: 4.

53. The method according to claim 48 wherein said contacting further comprises:
contacting the apolipoprotein B mRNA in the cell with a second chimeric protein comprising (i) a first polypeptide comprising a protein transduction domain and (ii) a second polypeptide comprising ACF or a fragment thereof which can bind to apolipoprotein B mRNA.

54. The method according to claim 53 wherein the second chimeric protein comprises an amino acid sequence of SEQ ID No: 6 or SEQ ID No: 8.

55. A method of reducing serum LDL levels comprising:
delivering into one or more cells of a patient, without genetically modifying the cells, an amount of a protein comprising APOBEC-1 or a fragment thereof which can edit mRNA encoding apolipoprotein B, which amount is effective to increase the concentration of VLDL-apolipoprotein B48 that is secreted by the one or more cells into serum and, consequently, reduce the serum concentration of LDL.

56. The method according to claim 55 wherein the one or more cells are liver cells, intestinal cells, or a combination thereof.

57. The method according to claim 55 wherein the patient is a mammal.

58. The method according to claim 57 wherein the mammal is a human.

59. The method according to claim 55 wherein said delivering comprises:
exposing the one or more cells to the protein under conditions effective to cause cellular uptake of the protein.

60. The method according to claim 59 wherein the protein is a chimeric protein which further comprises a polypeptide comprising a protein transduction domain.

61. The method according to claim 60 wherein the chimeric protein comprises an amino acid sequence of SEQ ID No: 2 or SEQ ID No: 4.

62. The method according to claim 59 wherein the protein is present in a liposome or niosome which is taken up by liver cells.

63. The method according to claim 55 wherein said delivering further comprises:
simultaneously delivering into the one or more cells of the patient, also without genetically modifying the cells, an amount of a second protein comprising ACF or a fragment thereof which can bind to apolipoprotein B mRNA.

64. The method according to claim 63 wherein said simultaneously delivering comprises:
exposing the one or more cells to the second protein under conditions effective to cause cellular uptake of the second protein.

65. The method according to claim 64 wherein the second protein is a chimeric protein which further comprises a polypeptide comprising a protein transduction domain.

66. The method according to claim 65 wherein the chimeric protein comprises an amino acid sequence of SEQ ID No: 6 or SEQ ID No: 8.

67. The method according to claim 55 further comprising:
repeating said delivering following a delay.

68. The method according to claim 67 wherein the delay is from about 1 to about 7 days.

69. A method of treating or preventing an atherogenic disease or disorder comprising:
administering to a patient an effective amount of a protein comprising APOBEC-1 or a fragment thereof which can edit mRNA encoding apolipoprotein B, wherein upon said administering the protein is taken up by one or more cells of the patient that can synthesize and secrete VLDL-apolipoprotein B under conditions which are effective to increase the concentration of VLDL-apolipoprotein B48 that is secreted by the one or more cells into serum, whereby rapid clearing of VLDL-apolipoprotein B48 from serum decreases the serum concentration of LDL to treat or prevent the atherogenic disease or disorder.

70. The method according to claim 69 wherein the patient is a mammal.

71. The method according to claim 70 wherein the mammal is a human.

72. The method according to claim 69 wherein said administering is carried out orally, topically, transdermally, parenterally, subcutaneously, intravenously, intramuscularly, intraperitoneally, by intracavitary or intravesical instillation, intraocularly, intraarterially, intralesionally, by application to mucous membranes, or by implantation.

73. The method according to claim 69 wherein the protein is a chimeric protein which further comprises a protein transduction domain.

74. The method according to claim 73 wherein the chimeric protein comprises an amino acid sequence of SEQ ID No: 2 or SEQ ID No: 4.

75. The method according to claim 69 wherein the polypeptide is present in a liposome or niosome which is taken up by liver cells.

76. The method according to claim 69 wherein said administering further comprises:
second administering to the patient an effective amount of a second protein comprising ACF or a fragment thereof which can bind to apolipoprotein B mRNA.

77. The method according to claim 76 wherein said second administering is carried out simultaneously.

78. The method according to claim 76 wherein the second polypeptide is a chimeric protein which further comprises a protein transduction domain.

79. The method according to claim 78 wherein the chimeric protein comprises an amino acid sequence of SEQ ID No: 6 or SEQ ID No: 8.

80. The method according to claim 69 further comprising:
repeating said administering following a delay.

81. The method according to claim 80 wherein the delay is from about 1 to about 7 days.

82. A liposome or niosome which is targeted for uptake by a liver cell, the liposome or niosome containing (i) APOBEC-1 or a fragment thereof which is effective to edit apolipoprotein B mRNA, (ii) ACF or a fragment thereof which is effective to bind apolipoprotein B mRNA, or (iii) a combination thereof.

83. The liposome or niosome according to claim 82 in the form of a liposome comprising asialofetuin incorporated into a lipid bilayer.

84. The liposome or niosome according to claim 82, in the form of a niosome comprising doxorubicin with a polyoxyethylene surface.

85. The liposome or niosome according to claim 82, wherein the liposome or niosome contains APOBEC-1 or a fragment thereof which is effective to edit apolipoprotein B mRNA.

86. The liposome or niosome according to claim 82, wherein the liposome or niosome contains ACF or a fragment thereof which is effective to bind apolipoprotein B mRNA.

87. The liposome or niosome according to claim 82, wherein the liposome or niosome contains a combination of APOBEC-1 or a fragment thereof which is effective to edit apolipoprotein B mRNA and ACF or a fragment thereof which is effective to bind apolipoprotein B mRNA.

88. A composition comprising:
a pharmaceutically acceptable carrier and the liposome or niosome according to claim 82.
